(19) United States
(12) Patent Application Publication
(10) Pub. No.: US 2021/0085775 A1
(43) Pub. Date: Mar. 25, 2021

(54) METHODS AND COMPOSITION FOR PREVENTING AND/OR TREATING CANCER

(71) Applicant: Valneva Sweden AB, Stockholm (SE)

(72) Inventors: Klaus Schwamborn, Nantes (FR); Tim Wood, Nacka (SE); Jianguang Ji, Malmö (SE); Jan Sundquist, Lund (SE); Kristina Sundquist, Lund (SE)

(73) Assignee: Valneva Sweden AB, Stockholm (SE)

(21) Appl. No.: 16/772,200

(22) PCT Filed: Sep. 3, 2018
(86) PCT No.: PCT/EP2018/000424

§ 371 Date: Jun. 12, 2020

(30) Foreign Application Priority Data
Dec. 15, 2017 (EP) EP17207685

(51) Int. Cl. A61K 39/02; A61K 39/39; A61K 39/395; C07K 16/22; A61P 35/00

(52) U.S. Cl. CPC A61K 39/107; A61K 39/39; A61K 2039/521; C07K 16/22; A61P 35/00; A61K 39/3955

(57) ABSTRACT

Described herein are compositions and methods for treating cancers, such as characterized by chronic inflammation or expression of GM1 ganglioside receptors

### FIELD

The disclosure relates to compositions and methods for treating cancer, such as cancer characterized by chronic inflammation or by expression of GM1 ganglioside receptors.

### BACKGROUND

Cancer survival depends on a number of factors, including the type and stage of cancer, the stage at diagnosis, extent of metastasis, and the age of the patient. For example, for prostate cancer, the survival rate is higher in patients who are diagnosed at a younger age and among those with a local or regional prostate cancer. The mean 5-year relative survival rate for patients with prostate cancer in Europe is around 83%; however, the survival rate ranges from 76 to 88%. For men diagnosed with prostate cancer, which has spread to other parts of the body, the 5-year survival rate is only 30%.

Colorectal cancer (CRC) is the third most common malignancy and the fourth most deadly cancer in the world (Arnold et al. Gut (2017) 66(4): 683-91). In the United States, CRC is the third leading cause of cancer-related deaths in women and the second leading cause in men (American Cancer Society: Cancer Facts & Figures (2017)). The incidence of CRC is lower in poor- and middle-income countries but appears to be increasing due to societal and economic development in these countries as well as a shift to Western diets and lifestyles. The incidence of CRC tends to be stabilizing or decreasing in developed nations, such as the United States and many European countries (Jemal et al. Cancer (2004) 101(1): 3-27; Ait Oukrim et al. BMJ (2015) 351: h4970); however, the incidence in these countries is still the highest in the world, most likely due to the high consumption of processed and unprocessed meat, which can lead to dysregulation of colonic microbial metabolism (Arnold et al. Gut (2017) 66(4): 683-91). In addition, the hygiene hypothesis which was thought to a causative factor in development of autoimmune and allergic diseases has now been linked to neoplasia (Oikonomopoulou et al. Clinical Cancer Research (2013) 19(11): 2834-41). Furthermore, an association between microorganisms, such as the intestinal microbiome, and CRC has been suggested (Baxter et al. Microbiome (2014) 2:20; Zackular et al. mBio (2013) 4(6): e00692-13; Zackular et al. mSphere (2016) 1(1); Belcheva et al. Cell (2014) 158(2): 288-99).

### SUMMARY

Cancer therapeutics for use in treating humans is a field where improved clinical options and solutions are in high need. Despite recent progress with immunomodulatory agents, alternative solutions and compositions, e.g. as an adjuvant type use, are important. Accordingly, new methods of treating and/or preventing cancer or enhancing survival of patients with cancer are desired.

The present disclosure provides methods of treating and/or preventing cancer involving administering compositions comprising lipopolysaccharide (LPS) from at least one strain of Vibrio cholerae, compositions comprising cholera toxin, or compositions comprising a combination of LPS from at least one strain of Vibrio cholerae and cholera toxin. Also provided herein are methods of enhancing survival of subjects having cancer involving administering compositions comprising lipopolysaccharide (LPS) from at least one strain of Vibrio cholerae, compositions comprising cholera toxin, or compositions comprising a combination of LPS from at least one strain of Vibrio cholerae and cholera toxin. We herein postulate that administration of the LPS in the context of cancer i.e., through stimulation of regulatory T cells in a subject in need thereof, and/or cholera toxin (e.g. cholera toxin subunit B)), results in a reduction of inflammation and a reduction in tumor growth and progression and is the dominating process that is able to provide an improvement to a subject who is at risk of developing or already has cancer. One of the ways to measure this improvement is by measuring the probability of survival (or probability of dying) of a subject within a group of such subjects who are at risk of developing or already have cancer (e.g., have been diagnosed with cancer).

Compositions for use in accordance with the methods described herein comprise a sufficient amount of LPS, cholera toxin, or a combination of LPS and cholera toxin, wherein the LPS may be any LPS including mimics of LPS. In some embodiments, the LPS is associated with inactivated or live-attenuated variants of a strain of Vibrio cholerae. Commercial products that may be used in subjects in need thereof are products with the tradenames Dukoral®, Shanchol®, Euvichol®, and/or Vaxchora®. Dosage strength may be a strength used for the vaccine but preferably may be higher or may be administered more frequently than currently done, i.e., may be administered three times or more (e.g., 3-, 4-, 5-, 6- or 7-times or more).

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE DRAWINGS

The accompanying drawings are not intended to be drawn to scale. The figures are illustrative only and are not required for enablement of the disclosure. For purposes of clarity, not every component may be labeled in every drawing. In the drawings:

FIG. 1 is an overview of the classification of the O1 and O139 serogroups that are associated with epidemic cholera disease.

FIG. 2 is a nonparametric estimate of the hazard ratio (HR) of prostate cancer mortality among patients with post-diagnostic use of cholera vaccine as a function of follow-up time. The figure indicates that the decreased HR was stronger after the vaccination of cholera vaccine, and it gradually became weaker after approximately 15 months.

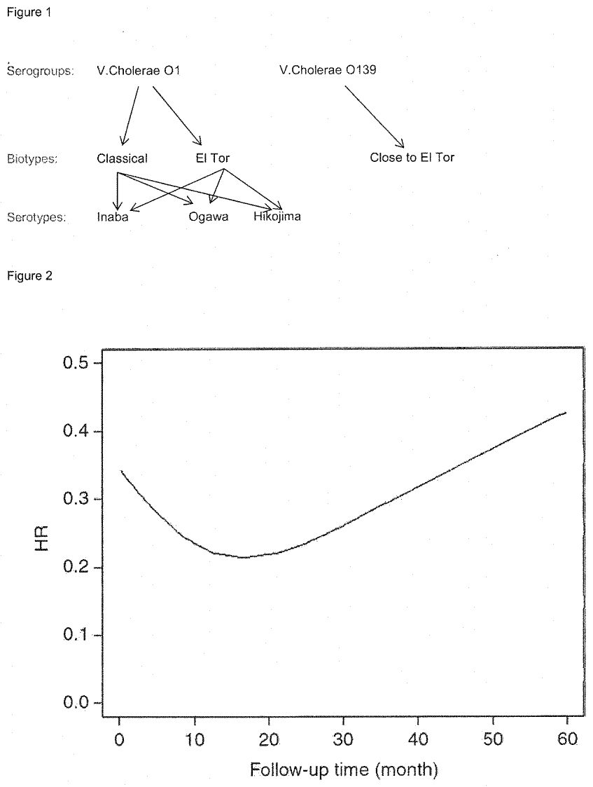

### DETAILED DESCRIPTION

Aspects of the present disclosure provide methods of treating and/or preventing cancer involving administering to a subject in need thereof a therapeutically effective amount of a composition comprising lipopolysaccharide from at least one strain of Vibrio cholerae, a composition comprising cholera toxin, or a composition comprising a combination of lipopolysaccharide from at least one strain of Vibrio cholerae and cholera toxin. Also provided herein are methods of enhancing survival of subjects having cancer by administering compositions comprising lipopolysaccharide from at least one strain of Vibrio cholerae, compositions comprising cholera toxin, or compositions comprising a combination of lipopolysaccharide from at least one strain of Vibrio cholerae and cholera toxin.

### Lipopolysaccharide

Lipopolysaccharide, also referred to as LPS, is a major component of the outer membrane of Gram-negative bacteria. Each LPS molecule is composed of a hydrophobic lipid section (lipid A), which is responsible for the toxic properties of the molecule; a hydrophilic core polysaccharide chain; and a repeating hydrophilic O-antigenic oligosaccharide side chain (O antigen) that is specific to the bacterial serotype.

LPS is generally considered to be a pro-inflammatory molecule, as the lipid A portion of LPS is the biologically active moiety that causes septic shock. The interaction of LPS with macrophages also results in the release of pro-inflammatory cytokines such as TNFα, IL-6, and IL-1.

Regulatory T cells (Tregs), characterized as CD4+, CD25 high and Foxp3+, downregulate immune responses to both foreign and self-antigens. A significant number of Tregs can be found in inflamed intestines, for example in chronic inflammatory disorders. Despite that LPS is typically associated with an acute pro-inflammatory effect and reports that LPS promotes liver metastasis in colorectal cancer by stimulating TLR4 signaling (Hsu Y C et al. Cancer Research (2011) 71(5): 1989f), it was reported also that exposure of LPS to particular cell types, such as CD4+CD25+ cells (e.g., Tregs), promotes survival and proliferation of these cells (Caramalho et al. J. Exp. Med. (2003) 197(4): 403-11; Lavelle et al. J. Leukocyte Biology (2004) 75:756f). Without wishing to be bound by any particular theory, we herein postulate that administration of the LPS (possibly in conjunction with the cholera toxin (e.g., cholera toxin subunit B)), in the context of cancer i.e., through stimulation of regulatory T cells in the subject results in a reduction of inflammation and a reduction in tumor growth and progression, and is the dominating process providing an improvement in the cancer disease setting and thus overall improves the probability of survival for a subject in need thereof, i.e. having (or is at risk of developing) cancer.

The phrase “LPS from at least one strain of V. cholerae” refers to LPS produced by V. cholerae, which may be in the form of LPS isolated from bacterial cells or LPS associated with V. cholerae cells, e.g., in the form of whole V. cholerae bacteria (inactivated strains (heat and/or formalin inactivated) and/or live attenuated strains) or outer membrane vesicles (OMVs) released by V. cholerae (Chatterjee D et al. Genetic Engineering & Biotechnology News (2011): 1357-1362). However, any type of LPS might be beneficial for the uses as disclosed herein, i.e. use in the treatment of cancer. A preferred embodiment is a composition comprising V. cholerae bacterial cells, e.g., in the form of whole V. cholerae bacteria (inactivated strains (heat and/or formalin inactivated) and/or live attenuated strains) that comprise LPS and/or cholera toxin such as subunit B (CTB) (see e.g., Chatterjee D et al. Genetic Engineering & Biotechnology News (2011): 1357-1362). Also within the definition of LPS from at least one strain of V. cholerae are mimitopes of LPS (i.e., LPS-mimicking peptides) as described by Ghazi et al. J. Pept. Sci. (2016): 22(11-12): 682-688) and others. More preferred embodiments are methods of treatment of (or uses in) colorectal cancer subjects in need thereof with compositions comprising inactivated O1 strains (of biotypes El Tor and/or classical; and subtypes Inaba and/or Ogawa inactivated by formalin and/or heat). Most preferred embodiments are methods of treatment of (or uses in) cancer subjects in need thereof with Cholera vaccines known in certain jurisdictions such as the US and the EU under the trademarks Dukoral®, Shanchol®, Euvichol® and/or Vaxchora®.

V. cholerae is a Gram-negative, curved rod-shaped bacterium with a polar flagellum. It is a facultative anaerobe and tends to tolerate alkaline media but is sensitive to acid (Finkelstein, Medical Microbiology “Cholera, Vibrio cholerae O1 and O139, and other Pathogenic Vibrios; 4^{th} Edition U.T. Medical Branch at Galveston (1996)). V. cholerae may be classified into distinct groups based on the structure of the O antigen of the LPS. In general, V. cholerae strains are classified as serogroup O1, serogroup O139, or non-O1/non-O139 based on agglutination of the bacterial cells (or lack thereof) in O1 and/or O139 antiserum. The non-O1/non-O139 strains have been divided into groups O2 through O138 based on the lipopolysaccharide (LPS) somatic (O) antigen. The majority of non-O1/non-O139 strains are not associated with cholera disease.

In some embodiments, the V. cholerae strain is V. cholerae O1. In some embodiments, the V. cholerae strain is V. cholerae O139. In some embodiments, the V. cholerae belongs to a non-O1 serogroup. Examples of non-O1 serogroups include the O2, O3, O4, O5, O6, O7, O8, O9, O10, O11, O12, O13, O14, O15, O16, O17, O18, O19, O20, O21, O22, O23, O24, O25, O26, O27, O28, O29, O30, O31, O32, O33, O34, O35, O36, O37, O38, O39, O40, O41, O42, O43, O44, O45, O46, O47, O48, O49, O50, O51, O52, O53, O54, O55, O56, O57, O58, O59, O60, O61, O62, O63, O64, O65, O66, O67, O68, O69, O70, O71, O72, O73, O74, O75, O76, O77, O78, O79, O80, O81, O82, O83, O84, O85, O86, O87, O88, O89, O90, O91, O92, O93, O94, O95, O96, O97, O98, O99, O100, O101, O102, O103, O104, O105, O106, O107, O108, O109, O110, O111, O112, O113, O114, O115, O116, O117, O118, O119, O120, O121, O122, O123, O124, O125, O126, O127, O128, O129, O130, O131, O132, O133, O134, O135, O136, O137, and O138 groups.

As will be evident to one of ordinary skill in the art, in some embodiments, the compositions described herein may contain LPS from strains of V. cholerae belonging to different O groups. In some embodiments, the compositions comprise LPS from one or more strains of V. cholerae O1 and one or more strains of V. cholerae belonging another O group.

The V. cholerae O1 group contains two major biotypes, El Tor and classical, each of which can be further distinguished into three serotypes based on the composition of the O antigen: Inaba, Ogawa, and Hikojima. Bacterial cells of each of the serotypes express the common “A” antigen; cells of the Ogawa serotype also express the “B” antigen i.e. express A+B antigens; cells of the Inaba serotype also express the “C” antigen, i.e. express A+C antigens; and cells of the Hikojima serotype express also the “B” and “C” antigens, i.e. express A+B+C antigens.

In some embodiments, the compositions described herein comprise LPS from at least one (e.g., 1, 2, 3, 4, 5, or more) strain belonging to V. cholerae O1 El Tor biotype. In some embodiments, the compositions described herein comprise LPS from at least one (e.g., 1, 2, 3, 4, 5, or more) strains belonging to V. cholerae O1 classical biotype. In some embodiments, the compositions described herein comprise LPS from at least one (e.g., 1, 2, 3, 4, 5, or more) strains belonging to V. cholerae O1 El Tor biotype and at least one (e.g., 1, 2, 3, 4, 5, or more) strains belonging to V. cholerae O1 classical biotype. In some embodiments, the compositions described herein comprise LPS from at least one (e.g., 1, 2, 3, 4, 5, or more) strain belonging to V. cholerae O1 El Tor biotype. In some embodiments, the compositions described herein comprise LPS from at least one (e.g., 1, 2, 3, 4, 5, or more) strains belonging to V. cholerae O1 classical Hikojima biotype. In some embodiments, the compositions described herein comprise LPS from at least one (e.g., 1, 2, 3, 4, 5, or more) strains belonging to V. cholerae O1 El Tor Hikojima biotype.

In some embodiments, the compositions described herein comprise a combination of LPS from at least one (e.g., 1, 2, 3, 4, 5, or more) strain belonging to V. cholerae O1 El Tor biotype and cholera toxin. In some embodiments, the compositions described herein comprise a combination of LPS from at least one (e.g., 1, 2, 3, 4, 5, or more) strains belonging to V. cholerae O1 classical biotype and cholera toxin. In some embodiments, the compositions described herein comprise a combination of LPS from at least one (e.g., 1, 2, 3, 4, 5, or more) strains belonging to V. cholerae O1 El Tor biotype and at least one (e.g., 1, 2, 3, 4, 5, or more) strains belonging to V. cholerae O1 classical biotype and cholera toxin. In some embodiments, the compositions described herein comprise a combination of LPS from at least one (e.g., 1, 2, 3, 4, 5, or more) strains belonging to V. cholerae O1 classical Hikojima biotype and cholera toxin. In some embodiments, the compositions described herein comprise a combination of LPS from at least one (e.g., 1, 2, 3, 4, 5, or more) strains belonging to V. cholerae O1 El Tor Hikojima biotype and cholera toxin.

In some embodiments, the compositions described herein comprise LPS from at least one (e.g., 1, 2, 3, 4, 5, or more) strain belonging to V. cholerae O1 El Tor biotype. In some embodiments, the compositions described herein comprise LPS from at least one (e.g., 1, 2, 3, 4, 5, or more) strains belonging to V. cholerae O1 classical biotype. In some embodiments, the compositions described herein comprise LPS from at least one (e.g., 1, 2, 3, 4, 5, or more) strain belonging to V. cholerae O1 El Tor and at least one (e.g., 1, 2, 3, 4, 5, or more) strain belonging to V. cholerae O1 classical biotype. In some embodiments, the compositions described herein comprise LPS from at least one (e.g., 1, 2, 3, 4, 5, or more) strain belonging to V. cholerae O139. In some embodiments, the compositions described herein comprise LPS from at least one (e.g., 1, 2, 3, 4, 5, or more) strain belonging to V. cholerae O1 El Tor and/or classical biotype and at least one (e.g., 1, 2, 3, 4, 5, or more) strain belonging to V. cholerae O139.

In some embodiments, the compositions described herein comprise LPS from at least one (e.g., 1, 2, 3, 4, 5, or more) strain belonging to V. cholerae O1 Inaba El Tor biotype. In some embodiments, the compositions described herein comprise LPS from at least one (e.g., 1, 2, 3, 4, 5, or more) strain belonging to V. cholerae O1 Ogawa El Tor biotype. In some embodiments, the compositions described herein comprise LPS from at least one (e.g., 1, 2, 3, 4, 5, or more) strain belonging to V. cholerae O1 Inaba classical biotype. In some embodiments, the compositions described herein comprise LPS from at least one (e.g., 1, 2, 3, 4, 5, or more) strain belonging to V. cholerae O1 Ogawa classical biotype.

In some embodiments, the compositions described herein comprise a combination of LPS from at least one (e.g., 1, 2, 3, 4, 5, or more) strain belonging to V. cholerae O1 El Tor biotype and cholera toxin. In some embodiments, the compositions described herein comprise LPS from at least one (e.g., 1, 2, 3, 4, 5, or more) strains belonging to V. cholerae O1 classical biotype and cholera toxin. In some embodiments, the compositions described herein comprise a combination of LPS from at least one (e.g., 1, 2, 3, 4, 5, or more) strain belonging to V. cholerae O1 El Tor and at least one (e.g., 1, 2, 3, 4, 5, or more) strain belonging to V. cholerae O1 classical biotype and cholera toxin. In some embodiments, the compositions described herein comprise LPS from at least one (e.g., 1, 2, 3, 4, 5, or more) strain belonging to V. cholerae O139. In some embodiments, the compositions described herein comprise LPS from at least one (e.g., 1, 2, 3, 4, 5, or more) strain belonging to V. cholerae O1 El Tor and/or classical biotype and at least one (e.g., 1, 2, 3, 4, 5, or more) strain belonging to V. cholerae O139 and cholera toxin.

In some embodiments, the compositions described herein comprise a combination of LPS from at least one (e.g., 1, 2, 3, 4, 5, or more) strain belonging to V. cholerae O1 Inaba El Tor biotype and cholera toxin. In some embodiments, the compositions described herein comprise a combination of LPS from at least one (e.g., 1, 2, 3, 4, 5, or more) strain belonging to V. cholerae O1 Ogawa El Tor biotype and cholera toxin. In some embodiments, the compositions described herein comprise a combination of LPS from at least one (e.g., 1, 2, 3, 4, 5, or more) strain belonging to V. cholerae O1 Inaba classical biotype and cholera toxin. In some embodiments, the compositions described herein comprise a combination of LPS from at least one (e.g., 1, 2, 3, 4, 5, or more) strain belonging to V. cholerae O1 Ogawa classical biotype and cholera toxin.

In some embodiments, the compositions described herein comprise LPS from at least two strains, wherein at least one of the strains belongs to V. cholerae El Tor biotype and at least one of the strains belongs to V. cholerae classical biotype. In some embodiments, the compositions described herein comprise LPS from at least two strains, wherein at least one of the strains belongs to V. cholerae Ogawa El Tor biotype and at least one of the strains belongs to V. cholerae Ogawa classical biotype. In some embodiments, the compositions described herein comprise LPS from at least two strains, wherein at least one of the strains belongs to V. cholerae Ogawa El Tor biotype and at least one of the strains belongs to V. cholerae Inaba classical biotype. In some embodiments, the compositions described herein comprise LPS from at least two strains, wherein at least one of the strains belongs to V. cholerae Ogawa El Tor biotype and at least one of the strains belongs to V. cholerae Inaba El Tor biotype. In some embodiments, the compositions described herein comprise LPS from at least two strains, wherein at least one of the strains belongs to V. cholerae Ogawa classical biotype and at least one of the strains belongs to V. cholerae Inaba classical biotype. In some embodiments, the compositions described herein comprise LPS from at least two strains, wherein at least one of the strains belongs to V. cholerae Ogawa classical biotype and at least one of the strains belongs to V. cholerae Inaba El Tor biotype. In some embodiments, the compositions described herein comprise LPS from at least two strains, wherein at least one of the strains belongs to V. cholerae Inaba classical biotype and at least one of the strains belongs to V. cholerae Inaba El Tor biotype.

In some embodiments, the compositions described herein comprise a combination of LPS from at least two strains and cholera toxin, wherein at least one of the strains belongs to V. cholerae El Tor biotype and at least one of the strains belongs to V. cholerae classical biotype. In some embodiments, the compositions described herein comprise a combination of LPS from at least two strains and cholera toxin, wherein at least one of the strains belongs to V. cholerae Ogawa El Tor biotype and at least one of the strains belongs to V. cholerae Ogawa classical biotype. In some embodiments, the compositions described herein comprise a combination of LPS from at least two strains and cholera toxin, wherein at least one of the strains belongs to V. cholerae Ogawa El Tor biotype and at least one of the strains belongs to V. cholerae Inaba classical biotype. In some embodiments, the compositions described herein comprise a combination of LPS from at least two strains and cholera toxin, wherein at least one of the strains belongs to V. cholerae Ogawa El Tor biotype and at least one of the strains belongs to V. cholerae Inaba El Tor biotype. In some embodiments, the compositions described herein comprise a combination of LPS from at least two strains and cholera toxin, wherein at least one of the strains belongs to V. cholerae Ogawa classical biotype and at least one of the strains belongs to V. cholerae Inaba classical biotype. In some embodiments, the compositions described herein comprise a combination of LPS from at least two strains and cholera toxin, wherein at least one of the strains belongs to V. cholerae Ogawa classical biotype and at least one of the strains belongs to V. cholerae Inaba El Tor biotype. In some embodiments, the compositions described herein comprise a combination of LPS from at least two strains and cholera toxin, wherein at least one of the strains belongs to V. cholerae Inaba classical biotype and at least one of the strains belongs to V. cholerae Inaba El Tor biotype.

In some embodiments, the compositions described herein comprise LPS from three strains of V. cholerae. In some embodiments, the compositions described herein comprise LPS from at least three strains, wherein at least one strain belongs to V. cholerae Ogawa El Tor biotype, at least one strain belongs to V. cholerae Ogawa classical biotype, and at least one strain belongs to V. cholerae Inaba classical biotype. In some embodiments, the compositions described herein comprise LPS from at least three strains, wherein at least one strain belongs to V. cholerae Ogawa El Tor biotype, at least one strain belongs to V. cholerae Inaba classical biotype, and at least one strain belongs to V. cholerae Inaba El Tor biotype. In some embodiments, the compositions described herein comprise LPS from at least three strains, wherein at least one strain belongs to V. cholerae Ogawa classical biotype, at least one strain belongs to V. cholerae Inaba classical biotype, and at least one strain belongs to V. cholerae Inaba El Tor biotype.

In some embodiments, the compositions described herein comprise a combination of LPS from three strains of V. cholerae and cholera toxin. In some embodiments, the compositions described herein comprise a combination of LPS from at least three strains and cholera toxin, wherein at least one strain belongs to V. cholerae Ogawa El Tor biotype, at least one strain belongs to V. cholerae Ogawa classical biotype, and at least one strain belongs to V. cholerae Inaba classical biotype. In some embodiments, the compositions described herein comprise a combination of LPS from at least three strains and cholera toxin, wherein at least one strain belongs to V. cholerae Ogawa El Tor biotype, at least one strain belongs to V. cholerae Inaba classical biotype, and at least one strain belongs to V. cholerae Inaba El Tor biotype. In some embodiments, the compositions described herein comprise a combination of LPS from at least three strains and cholera toxin, wherein at least one strain belongs to V. cholerae Ogawa classical biotype, at least one strain belongs to V. cholerae Inaba classical biotype, and at least one strain belongs to V. cholerae Inaba El Tor biotype.

In some embodiments, the compositions described herein comprise LPS from four strains of V. cholerae. In some embodiments, the compositions described herein comprise LPS from five strains of V. cholerae. In some embodiments, the compositions described herein comprise LPS from six or more strains of V. cholerae.

In some embodiments, the compositions described herein comprise a combination of LPS from four strains of V. cholerae and cholera toxin. In some embodiments, the compositions described herein comprise a combination of LPS from five strains of V. cholerae and cholera toxin. In some embodiments, the compositions described herein comprise a combination of LPS from six or more strains of V. cholerae and cholera toxin.

In some embodiments, the compositions comprise LPS derived from or obtained from V. cholerae. For example, in some embodiments, the LPS may be isolated or separated from the V. cholerae bacteria. Methods of obtaining LPS from bacteria are known in the art, for example, the rapid isolation method, which involves gas chromatography and mass spectroscopy (see, e.g., Yi et al. Analyst. 2000 April; 125(4):651-6), and hot aqueous-phenol extraction (see, e.g., Davis et al. J Vis Exp. (2012)(63): 3916). In some embodiments, the compositions comprise LPS derived from or obtained from V. cholerae as described previously and cholera toxin derived from or obtained from V. cholerae.

In some embodiments, the compositions described herein comprise LPS that is the form of whole V. cholerae bacteria. As used herein, the term “whole V. cholerae bacteria” refers to a population of bacteria that are substantially intact bacteria. In some embodiments, the whole V. cholerae bacteria have not been subjected to a process of bacteriolysis or have not been separated into distinct fractions or components. As will be appreciated by one of ordinary skill in the art, whole V. cholerae bacteria may include a portion of bacteria that are not in whole bacterial form, such as a portion of bacteria that have lysed. In some embodiments, the whole V. cholerae bacteria does not contain a substantial amount of lysed bacteria. In some embodiments, at least 50%, 55%, 60%, 65%, 70%, 75%, 80%, 85%, 90%, 95%, or up to 100% of the bacteria of the whole V. cholerae bacteria are in whole bacterial form (e.g., not lysed or fractionated).

Methods for quantifying the amount of whole bacteria in a composition are known in the art and include microscopy methods and assays for detecting bacterial components (e.g., nucleic acid, cytoplasmic components) indicative that the bacteria are not in whole bacterial form.

In some embodiments, the compositions described herein comprise LPS from more than one (e.g., 2, 3, 4, 5, or more) V. cholerae strain. In some embodiments, the compositions described herein comprise a combination of LPS from more than one (e.g., 2, 3, 4, 5, or more) V. cholerae strain and cholera toxin. In such embodiments, as will be appreciated by one of ordinary skill in the art, the LPS from each of the V. cholerae strains may be in the same or different form. For example, in some embodiments, the composition may comprise LPS from more than one V. cholerae strain, in which the LPS from each of the V. cholerae strains has been isolated from the V. cholerae bacteria. In some embodiments, the composition may comprise LPS from more than one V. cholerae strain, in which the LPS from each of the V. cholerae strains is present in the form of whole V. cholerae bacteria, meaning the composition comprises whole bacteria of more than one V. cholerae strain. In some embodiments, the composition may comprise LPS from more than one V. cholerae strain, in which the LPS from one (or more) of the V. cholerae strains has been isolated from the V. cholerae bacteria and the LPS from one (or more) of the V. cholerae strains is present in the form of whole V. cholerae bacteria.

In some embodiments, the whole V. cholerae bacteria are killed or inactivated bacteria. In some embodiments, the whole V. cholerae bacteria are subjected to a process by which the bacteria is rendered dead or metabolically inactive. A variety of methods of killing or inactivating bacteria are known in the art. In some embodiments, the bacteria are inactivated by chemical inactivation, thermal inactivation, pH inactivation, ionizing radiation inactivation, or UV inactivation. The viability or activity of the bacteria following the process of killing or inactivation may be assessed, for example by viability staining or plating on growth medium.

In some embodiments, the whole V. cholerae bacteria are killed or inactivated by thermal inactivation. In some embodiments, the whole V. cholerae bacteria are heat inactivated. In general, heat inactivation of bacteria involves subjecting the bacteria to elevated temperatures for a duration of time sufficient to inactivate the bacteria or eliminate viability.

In some embodiments, the whole V. cholerae bacteria are killed or inactivated by chemical inactivation. Examples of chemical agents for use in chemically inactivating bacteria include, without limitation, formalin, alcohols, salt, antibiotics, and detergents. In some embodiments, the whole V. cholerae bacteria are chemically inactivated. In general, chemical inactivation of bacteria involves subjecting the bacteria to a chemical agent (e.g., formalin) under conditions for a period of time sufficient to inactivate the bacteria or eliminate viability. In some embodiments, the whole V. cholerae bacteria are formalin-inactivated.

As will be appreciated by one of ordinary skill in the art, in some embodiments, each of the V. cholerae strains of a composition may be inactivated by the same or different method. For example, in some embodiments, the composition may comprise V. cholerae bacteria that have been thermally-inactivated. In some embodiments, the composition may comprise V. cholerae bacteria that have been heat-inactivated. In some embodiments, the composition may comprise at least one V. cholerae strain that has been heat-inactivated. In some embodiments, each of the V. cholerae strains of the composition have been heat-inactivated. In some embodiments, the composition may comprise V. cholerae bacteria that have been chemically inactivated. In some embodiments, the composition may comprise V. cholerae bacteria that have been formalin inactivated. In some embodiments, the composition may comprise at least one V. cholerae strain that has been formalin-inactivated. In some embodiment, each of the V. cholerae strains of the composition have been formalin-inactivated.

In some embodiments, the composition may comprise bacteria that have been heat-inactivated and bacteria that have been formalin-inactivated. In some embodiments, the composition may comprise bacteria of a V. cholerae strain that has been heat-inactivated and bacteria of the same V. cholerae strain that has been formalin-inactivated. In some embodiments, each of the V. cholerae strains have been inactivated using the same method.

In some embodiments, the whole V. cholerae bacteria are live, attenuated V. cholerae bacteria, such as lyophilized V. cholerae CVD 103-HgR (Chen et al. Clinical Infectious Diseases (2016) 62: 1329f).

In some embodiments, the composition comprises V. cholerae O1 Inaba, classical biotype; V. cholerae O1 Inaba, El Tor biotype; V. cholerae O1 Ogawa, classical biotype; and cholera toxin. In some embodiments, the composition comprises V. cholerae O1 Inaba, classical biotype; V. cholerae O1 Inaba, El Tor biotype; V. cholerae O1 Ogawa, classical biotype; and cholera toxin subunit B. In some embodiments, the composition comprises heat-inactivated V. cholerae O1 Inaba, classical biotype; formalin-inactivated V. cholerae O1 Inaba, El Tor biotype; heat-inactivated V. cholerae O1 Ogawa, classical biotype; formalin-inactivated V. cholerae O1 Ogawa, classical biotype; and recombinant cholera toxin subunit B. In some embodiments, the composition is the cholera vaccine, Dukoral®. Examples of compositions comprising LPS and cholera toxin are known in the art, e.g., Dukoral®, Vaxchora®, Shanchol® and/or Euvichol®. In some embodiment, the composition is the cholera vaccine, Dukoral® as e.g. described in PCT Publication WO 2011/034495A1, summary of product characteristics of Dukoral® provided by EMA. In some embodiments, the composition is the cholera vaccine, Vaxchora®. Examples of compositions comprising LPS and cholera toxin are known in the art, e.g., summary of product characteristics of Vaxchora® provided by the FDA. In some embodiment, the composition is the cholera vaccine Shanchol®, as e.g. described in the officially approved packet inserts in licensed countries. In some embodiment, the compositions is the cholera vaccine Euvichol®, as e.g. described in the officially approved packet inserts in licensed countries.

In short, the above-mentioned four cholera vaccines contain the active ingredients as listed in Tables A-C.

TABLE A
Active ingredients and quantity of Dukoral ®:

| Active Ingredients | Quantity |
|---|---|
| Vibrio cholerae O1 Inaba, | 31.25 × 10⁹ bacteria |
| classical biotype | |
| (heat-inactivated) | |
| Vibrio cholerae O1 Inaba, | 31.25 × 10⁹ bacteria |
| El Tor biotype | |
| (formalin-inactivated) | |
| Vibrio cholerae O1 | 31.25 × 10⁹ bacteria |
| Ogawa, classical | |
| biotype (heat-inactivated) | |
| Vibrio cholerae O1 | 31.25 × 10⁹ bacteria |
| Ogawa, classical | |
| biotype (formalin-inactivated) | |
| Recombinant cholera | 1 mg |
| toxin B subunit | |
| (rCTB) | |

TABLE B
Active, ingredients and quantity of Vachora ®:

| Active Ingredients | Quantity |
|---|---|
| Lyophilized V. cholerae CVD | After reconstitution, |
| 103-HgR (CVD 103-HgR | 4 × 10⁸ to 2 × 10⁹ |
| was constructed from the serogroup | colony forming units |
| O1 classical Inaba strain 569B by | (CFU) of live attenuated |
| deleting the catalytic domain | V. cholerae CVD 103-HgR |
| sequence of both copies of the ctxA gene, | |
| which prevents the synthesis of active | |
| cholera toxin (CT). This attenuated strain | |
| remains able to synthesize the | |
| immunogenic non-toxic B subunit | |
| of CT (encoded by the ctxB gene). | |
| In addition, a marker was inserted into | |
| the hemolysin gene locus (hlyA) to | |
| enable differentiation of the | |
| vaccine strain from wild type | |
| V. cholerae O1.) | |

TABLE C
Active ingredients and quantity of Shanchol ® and Euvichol ®:

| Active Ingredients | Quantity |
|---|---|
| V. cholerae O1 Inaba | 300 Lipopolysaccharide |
| Cairo 48 classical | ELISA Units (L.E.U.) |
| biotype, Heat inactivated | |
| V. cholerae O1 Inaba | 600 L.E.U. |
| Phil 6973 El Tor | |
| biotype, Formalin | |
| inactivated | |
| V. cholerae O1 Ogawa | 300 L.E.U. |
| Cairo 50 classical | |
| biotype, Formalin | |
| inactivated | |
| V. cholerae O1 | 300 L.E.U. |
| Ogawa Cairo 50 | |
| classical biotype, | |
| Heat inactivated | |
| V. cholerae O139 | 600 L.E.U. |
| 4260B, Formalin | |
| inactivated | |

### Cholera Toxin

Aspects of the present disclosure provide methods of treating cancer comprising administering compositions comprising cholera toxin or compositions comprising a combination of LPS from at least one strain of Vibrio cholerae and cholera toxin. Also provided are methods of enhancing survival of asubject having cancer comprising administering compositions comprising cholera toxin or compositions comprising a combination of LPS from at least one strain of Vibrio cholerae and cholera toxin.

Cholera toxin is the main virulence factor produced by the CTXϕ bacteriophage residing in V. cholerae. Cholera toxin is composed of six protein subunits: a single copy of the A subunit and five copies of the B subunit. During infection with V. cholerae, the B subunit ring of the cholera toxin binds to target cells and the entire toxin complex is endocytosed by the cell, leading to release of the cholera toxin A subunit. Subunit B of cholera toxins is not toxic alone. Cholera toxin binds to human cells through interaction between the cholera toxin B subunit with GM1 ganglioside receptors on the cell surface.

Cholera toxin subunit B has adjuvant activity for mucosal vaccine; this may be due to the enhanced antigen presentation by various types of antigen-presenting cells, such as macrophages and dendritic cells (Bharati et al. Indian J. Med. Res. (2011) 133: 179-187; Baldauf et al. Toxins (2015) 7: 974-996) In addition to its adjuvant properties, cholera toxin subunit B may as an anti-inflammatory agent by modulating specific signal transduction pathways and may function as an immunomodulatory agent for treatment of various autoimmune disorders (Bharati et al. Indian J. Med. Res. (2011) 133: 179-187; Baldauf et al. Toxins (2015) 7: 974-996). Oral administration of cholera toxin can upregulate the accumulation of macrophages, natural killer (NK) cells, and the regulatory T cells, as well as IL-10 production, and can downregulate the accumulation of neutrophils (Doulberis et al. Carcinogenesis (2015) 280-290).

Administration of cholera toxin in the context of inflammation-associated carcinogenesis models has been shown to reduce tumor formation (see, e.g., Poutahidis et al. Oncolmmunology (2015) 4:10, e1027474 and Doulberis et al. Carcinogenesis (2015) 36(2): 280-90). Additionally, use of cholera toxin as an anti-inflammatory agent to treat inflammatory disease has been proposed (see, e.g., Royal et al. Toxins (2017) 9: 379).

The immunomodulatory function of CTB may be due to its specific properties, such as the ability of binding to specific GM1 ganglioside receptors present in the gut mucosa, and facilitating antigen uptake and presentation. Previous studies have found that MAPK phosphatase-1 expression can be induced by CTB alone and can subsequently inhibit the activation of Janus kinase and p38, thus leading to a substantial attenuation of TNFα and IL-6 production from macrophages (Chen et al. J. Immunol. (2002) 169:6408-6416). It was hypothesized that the immune function in prostate cancer patients might be altered when they receive a composition comprising cholera toxin, such as cholera vaccination that includes recombinant cholera toxin B subunit.

In some embodiments, the compositions described herein comprise a subunit of cholera toxin. In some embodiments, the compositions described herein comprise subunit B of cholera toxin. In some embodiments, the compositions described herein comprise a combination of LPS and a subunit of cholera toxin. In some embodiments, the compositions described herein comprise a combination of LPS and subunit B of cholera toxin.

As discussed in Baldauf et al. (Toxins (2015) 7: 974-996) the presence of cholera toxin or the presence of subunit A of cholera toxin in a preparation of subunit B of cholera toxin may affect one or more effect (e.g., stimulation of TNFα) of subunit B of cholera toxin. In some embodiments, the compositions described herein do not comprise subunit A of cholera toxin. In some embodiments, the compositions described herein do not comprise a detectable amount of subunit A of cholera toxin. In some embodiments, the compositions described herein do not comprise a substantial amount of subunit A of cholera toxin.

In some embodiments, any of the compositions described herein comprise subunit B of cholera toxin that is at least 95.0%, 95.5%, 96.0%, 96.5%, 97.0%, 97.5%, 98.0%, 98.5%, 99.0%, 99.1%, 99.2%, 99.3%, 99.4%, 99.5%, 99.6%, 99.7%, 99.8%, 99.9% pure. In some embodiments, any of the compositions described herein comprise subunit B of cholera toxin and less than 0.50%, 0.045%, 0.40%, 0.35%, 0.30%, 0.25%, 0.20%, 0.15%, 0.10%, 0.05%, 0.01% cholera toxin and/or subunit A of cholera toxin.

Cholera toxin, including subunits of cholera toxin, can be obtained by any method known in the art. For example, cholera toxin or subunits thereof may be isolated from V. cholerae strains that produce the toxin. In some embodiments, cholera toxin or subunits thereof may be recombinantly produced, for example by expressing the toxin or toxin subunit in a cell or expression system. In some embodiments, the cholera toxin subunit B is recombinantly produced. In some embodiments, the cholera toxin or subunits thereof may be recombinantly produced and added to a composition comprising LPS. In some embodiments, the cholera toxin or subunits thereof may be present in the LPS, for example in LPS obtained from V. cholerae. In some embodiments, the LPS is in the form of whole V. cholerae bacteria.

In some embodiments, as will be appreciated by one of ordinary skill in the art, the LPS and cholera toxin may be obtained from or derived from the same V. cholerae strain or different V. cholerae strains.

Also with the scope of the present disclosure are cholera toxin subunit B variants and cholera toxin subunit A variants. As used herein, the term “cholera toxin subunit B variant” or “cholera toxin subunit A variant” refers to a cholera toxin subunit B or cholera toxin subunit A having at least one amino acid mutation (e.g., insertion, deletion, substitution) relative to the amino acid sequence of a wild type or naturally occurring cholera toxin subunit B or cholera toxin subunit A.

As discussed herein, V. cholerae may be classified into distinct groups based on the structure of the O antigen of the LPS. In some embodiments, the cholera toxin is from a V. cholerae strain that is of serogroup V. cholerae O1. In some embodiments, the cholera toxin is from a V. cholerae strain that is of serogroup V. cholerae O139. In some embodiments, the cholera toxin is from a V. cholerae strain that belongs to a non-O1 serogroup. Examples of non-O1 serogroups include the O2, O3, O4, O5, O6, O7, O8, O9, O10, O11, O12, O13, O14, O15, O16, O17, O18, O19, O20, O21, O22, O23, O24, O25, O26, O27, O28, O29, O30, O31, O32, O33, O34, O35, O36, O37, O38, O39, O40, O41, O42, O43, O44, O45, O46, O47, O48, O49, O50, O51, O52, O53, O54, O55, O56, O57, O58, O59, O60, O61, O62, O63, O64, O65, O66, O67, O68, O69, O70, O71, O72, O73, O74, O75, O76, O77, O78, O79, O80, O81, O82, O83, O84, O85, O86, O87, O88, O89, O90, O91, O92, O93, O94, O95, O96, O97, O98, O99, O100, O101, O102, O103, O104, O105, O106, O107, O108, O109, O110, O111, O112, O113, O114, O115, O116, O117, O118, O119, O120, O121, O122, O123, O124, O125, O126, O127, O128, O129, O130, O131, O132, O133, O134, O135, O136, O137, and O138 groups.

As will be evident to one of ordinary skill in the art, in some embodiments, the compositions described herein may contain cholera toxin from strains of V. cholerae belonging to different O groups. In some embodiments, the compositions comprise cholera toxin from one or more strains of V. cholerae O1 and one or more strains of V. cholerae belonging another O group.

In some embodiments, the compositions described herein comprise cholera toxin from more than one (e.g., 2, 3, 4, 5, or more) V. cholerae strain. In some embodiments, the compositions described herein comprise cholera toxin from at least one (e.g., 1, 2, 3, 4, 5, or more) strain belonging to V. cholerae O1 Inaba classical biotype. In some embodiments, the compositions described herein comprise cholera toxin from at least one (e.g., 1, 2, 3, 4, 5, or more) strain belonging to V. cholerae O1 Inaba El Tor biotype. In some embodiments, the compositions described herein comprise cholera toxin from at least one (e.g., 1, 2, 3, 4, 5, or more) strain belonging to V. cholerae O1 Ogawa classical biotype. In some embodiments, the compositions described herein comprise cholera toxin from at least one (e.g., 1, 2, 3, 4, 5, or more) strain belonging to V. cholerae O1 Inaba El Tor biotype. In some embodiments, the compositions described herein comprise cholera toxin from at least one (e.g., 1, 2, 3, 4, 5, or more) strain belonging to V. cholerae O1 Hikojima classical biotype. In some embodiments, the compositions described herein comprise cholera toxin from at least one (e.g., 1, 2, 3, 4, 5, or more) strain belonging to V. cholerae O1 Hikojima El Tor biotype.

In some embodiments, the compositions described herein comprise cholera toxin from at least two strains, wherein at least one of the strains belongs to V. cholerae El Tor biotype and at least one of the strains belongs to V. cholerae classical biotype. In some embodiments, the compositions described herein comprise cholera toxin from at least two strains, wherein at least one of the strains belongs to V. cholerae Ogawa El Tor biotype and at least one of the strains belongs to V. cholerae Ogawa classical biotype. In some embodiments, the compositions described herein comprise cholera toxin from at least two strains, wherein at least one of the strains belongs to V. cholerae Ogawa El Tor biotype and at least one of the strains belongs to V. cholerae Inaba classical biotype. In some embodiments, the compositions described herein comprise cholera toxin from at least two strains, wherein at least one of the strains belongs to V. cholerae Ogawa El Tor biotype and at least one of the strains belongs to V. cholerae Inaba El Tor biotype. In some embodiments, the compositions described herein comprise cholera toxin from at least two strains, wherein at least one of the strains belongs to V. cholerae Ogawa classical biotype and at least one of the strains belongs to V. cholerae Inaba classical biotype. In some embodiments, the compositions described herein comprise cholera toxin from at least two strains, wherein at least one of the strains belongs to V. cholerae Ogawa classical biotype and at least one of the strains belongs to V. cholerae Inaba El Tor biotype. In some embodiments, the compositions described herein comprise cholera toxin from at least two strains, wherein at least one of the strains belongs to V. cholerae Inaba classical biotype and at least one of the strains belongs to V. cholerae Inaba El Tor biotype.

In some embodiments, the compositions described herein comprise cholera toxin from three strains of V. cholerae. In some embodiments, the compositions described herein comprise cholera toxin from at least three strains, wherein at least one strain belongs to V. cholerae Ogawa El Tor biotype, at least one strain belongs to V. cholerae Ogawa classical biotype, and at least one strain belongs to V. cholerae Inaba classical biotype. In some embodiments, the compositions described herein comprise cholera toxin from at least three strains, wherein at least one strain belongs to V. cholerae Ogawa El Tor biotype, at least one strain belongs to V. cholerae Inaba classical biotype, and at least one strain belongs to V. cholerae Inaba El Tor biotype. In some embodiments, the compositions described herein comprise cholera toxin from at least three strains, wherein at least one strain belongs to V. cholerae Ogawa classical biotype, at least one strain belongs to V. cholerae Inaba classical biotype, and at least one strain belongs to V. cholerae Inaba El Tor biotype.

In some embodiments, the compositions described herein comprise cholera toxin from a strain belonging to V. cholerae O1 Inaba classical biotype, a strain belonging to V. cholerae O1 Inaba El Tor biotype, and/or a strain belonging to V. cholerae O1 Ogawa classical biotype.

In some embodiments, the compositions described herein comprise cholera toxin from four strains of V. cholerae. In some embodiments, the compositions described herein comprise cholera toxin from five strains of V. cholerae. In some embodiments, the compositions described herein comprise cholera toxin from six or more strains of V. cholerae.

Methods of obtaining cholera toxin from bacteria are known in the art, for example, utilizing crossflow microfiltration followed by ion exchange chromatography (see, e.g., Jang et al, J Microbiol Biotechnol. 2009 January; 19(1):108-112), and fractionation onto two successive phosphocellulose columns (see, e.g., Mekalanos et al. Infect Immun. 1978 May; 20(2): 552-558).

In some embodiments, the compositions described herein comprise cholera toxin that is associated with whole V. cholerae bacteria. As used herein, the term “whole V. cholerae bacteria” refers to a population of bacteria that are substantially intact bacteria. In some embodiments, the whole V. cholerae bacteria have not been subjected to a process of bacteriolysis or have not been separated into distinct fractions or components.

In some embodiments, the composition comprises cholera toxin from strains belonging to V. cholerae O1 Inaba classical biotype; V. cholerae O1 Inaba, El Tor biotype; and V. cholerae O1 Ogawa classical biotype. In some embodiments, the composition comprises subunit B of cholera toxin from strains belonging to V. cholerae O1 Inaba classical biotype; V. cholerae O1 Inaba, El Tor biotype; and V. cholerae O1 Ogawa classical biotype. In some embodiments, the composition comprises recombinant cholera toxin having the same amino acid sequence as cholera toxin from strains belonging to V. cholerae O1 Inaba classical biotype; V. cholerae O1 Inaba, El Tor biotype; and V. cholerae O1 Ogawa classical biotype. In some embodiments, the composition is the cholera vaccine, Dukoral®. Examples of compositions comprising cholera toxin are known in the art, e.g., Dukoral®, Vaxchora®, Shanchol® and/or Euvichol®. In some embodiment, the composition is the cholera vaccine, Dukoral® as e.g. described in PCT Publication WO 2011/034495A1, summary of product characteristics of Dukoral® provided by EMA. In some embodiments, the composition is the cholera vaccine, Vaxchora®. Examples of compositions comprising cholera toxin are known in the art, e.g., summary of product characteristics of Vaxchora® provided by the FDA. In some embodiment, the composition is the cholera vaccine Shanchol, as e.g. described in the officially approved packet inserts in licensed countries. In some embodiment, the compositions is the cholera vaccine Euvichol®, as e.g. described in the officially approved packet inserts in licensed countries.

### Methods of Administration

Provided herein are methods for administering compositions comprising LPS from at least one V. cholerae strain, compositions comprising cholera toxin, or compositions comprising a combination of LPS from at least one V. cholerae strain and cholera toxin from at least one V. cholerae strain to a subject in need thereof. As used herein, a “subject,” “individual,” and “patient” are used interchangeably, and refer to a vertebrate, preferably a mammal. In some embodiments, the subject is a mammalian subject, such as a human, non-human primate, rodent, rabbit, sheep, dog, cat, horse, or cow. In some embodiments, the subject is a human subject, such as a patient.

The presence of another disease or disorder may predispose a subject to having or being at risk of having a cancer, such as a cancer characterized by chronic inflammation or by expression of GM1 ganglioside receptors. In some embodiments, a subject may be at risk of having cancer if the subject has a chronic inflammatory disorder. In some embodiments, a subject may be at risk of having cancer if the subject expresses GM1 ganglioside receptors.

In some embodiments, the subject has cancer or is predisposed to (at risk of) developing cancer. Examples of cancer include, without limitation, colorectal cancer, carcinoma, glioma, mesothelioma, melanoma, lymphoma, leukemia, adenocarcinoma, breast cancer, ovarian cancer, cervical cancer, glioblastoma, multiple myeloma, prostate cancer, Burkitt's lymphoma, head and neck cancer, colon cancer, non-small cell lung cancer, small cell lung cancer, cancer of the esophagus, stomach cancer, pancreatic cancer, hepatobiliary cancer, cancer of the gallbladder, cancer of the small intestine, rectal cancer, kidney cancer, bladder cancer, prostate cancer, penile cancer, urethral cancer, testicular cancer, vaginal cancer, uterine cancer, thyroid cancer, parathyroid cancer, adrenal cancer, pancreatic endocrine cancer, carcinoid cancer, bone cancer, skin cancer, retinoblastomas, Hodgkin's lymphoma, non-Hodgkin's lymphoma, Kaposi's sarcoma, multicentric Castleman's disease, AIDS-associated primary effusion lymphoma, neuroectodermal tumors, or rhabdomyosarcoma. In some embodiments of the methods provided herein, the cancer is colorectal cancer, prostate cancer, bladder cancer, small cell lung cancer, renal cancer, cervical cancer, or lymphoma.

In some embodiments, the cancer is associated with or characterized by chronic inflammation. Inflammation is a condition caused by the body's immune system as a response to injury or infection. Non-limiting symptoms associated inflammation include increased blood flow to the injury or infection site, leading redness and swelling, increased white blood cell production, and increased body temperature in the subject. Inflammation can be categorized as either acute or chronic. Acute inflammation typically persists for only a few days to a few weeks. However, chronic inflammation may be long-term or reoccurring, and can last from several months or longer. Chronic inflammation may result, for example, from failure to eliminate the cause of acute inflammation, an autoimmune response to a self-antigen, or a chronic irritant of low intensity that persists. Chronic inflammation has been linked to numerous illnesses, including cancer. In some embodiments, the cancer is associated with or characterized by chronic inflammation.

Chronic inflammation is a critical component in tumor progression. The tumor microenvironment, which is largely controlled by inflammatory cells, is an indispensable participant in the neoplastic process, fostering proliferation, survival, and migration of tumors. In addition, tumors have co-opted some of the signaling molecules present in the innate immune system, such as selection, chemokines, and their receptors for invasion, migration, and metastasis. Furthermore, numerous cancers have been linked to specific chronic inflammation conditions, including but not limited to, Helicobacter pylori infections leads to gastric cancer and lymphoma; papilloma and hepatitis viruses leading to cervical and liver carcinoma, respectively; autoimmune diseases, such as inflammatory bowel disease and Crohn's disease, which may lead to colorectal cancer; and inflammatory conditions of uncertain etiology, such as prostatitis, which may lead to prostate cancer (Colotta et al, Carcinogenesis (2009) 30(70): 1073-81).

In some embodiments, the cancer is associated with or characterized by expression of GM1 ganglioside receptors. Gangliosides are amphipathic molecules organized in the plasma membrane, with their hydrophobic moiety contained within the lipid bilayer and the hydrophilic moiety oriented to the extracellular environment. Gangliosides are typically thought to act as receptors for toxins (e.g., cholera toxin, as described herein), hormones, and other bioactive factors. GM1 ganglioside receptors are expressed in cells throughout the human body, such as intestinal epithelial cells, neurons, and immune cells. As discussed herein, subunit B of cholera toxin binds to GM1 ganglioside receptors. Examples of cancers characterized by expression of GM1 ganglioside receptors include, without limitation, colorectal cancer, prostate cancer, bladder cancer, small cell lung cancer, renal cancer, cervical cancer, and lymphoma. In some embodiments, the cancer is prostate cancer. In some embodiments, the cancer is colorectal cancer.

In some embodiments, the subject has or is predisposed to have cancer, such as cancer characterized by chronic inflammation or expression of GM1 ganglioside receptors. In some embodiments, the subject has or is at risk of having cancer, such as cancer characterized by chronic inflammation or expression of GM1 ganglioside receptors.

Whether a subject has a cancer or is at risk of having a cancer will be evident to one of skill in the art and may be determined by performing an assessment, such as, but not limited to, a prostate-specific antigen measurement, a cystoscopy or urine cytology, a colonoscopy, a computed tomography (CT) scan, cytology (e.g., Pap smear), or histopathology of lymph nodes. In some embodiments, the subject has one or more symptoms associated with cancer, such as difficult or painful urination, the presence of polyps in the colon, blood in the urine, chest pain, bleeding between menstrual cycles, or enlarged lymph nodes.

In some embodiments, the cancer is colorectal cancer. In general, colorectal cancer is a cancer that begins within the colon or rectum and is associated with the present of colon polyps. Colorectal cancers include adenocarcinomas, carcinoid tumors, gastrointestinal stromal tumors, lymphomas, and sarcomas. Risk factors associated with increased incidence of having or developing colorectal cancer include, for example, western style dietary habits, obesity, tobacco-smoking, and lack of physical activity (Janakiram et al. Adv. Exp. Med. Biol. (2014) 816:25-52).

In some embodiments, the subject has colorectal cancer or is at risk of having colorectal cancer. Whether a subject has colorectal cancer or at risk of colorectal cancer will be evident to one of skill in the art and may be determined by performing an assessment, such as a colonoscopy, fecal occult blood testing, and sigmoidoscopy. In some embodiments, the subject has one or more symptoms associated with colorectal cancer, such as blood in the stool, rectal bleeding, cramping, change in bowel movements, and weight loss.

The presence of another disease or disorder may predispose a subject to having or being at risk of having colorectal cancer. In some embodiments, a subject may be at risk of having colorectal cancer if the subject has a chronic inflammatory disorder. For example, an increase in the risk of colorectal cancer has be found in individuals with chronic inflammation or an inflammatory disorder, such as inflammatory bowel disease (see, e.g., Triantafillidis et al. Anticancer Res. (2009) 29(7): 2727-37; Terzic et al. Gastroenterology (2010) 138(6): 2101-2114). In some embodiments, the subject has an inflammatory bowel disease, such as ulcerative colitis or Crohn's disease.

In some embodiments, the cancer is prostate cancer. Prostate cancer is a cancer that begins within the prostate gland. Risk factors associated with increased incidence of having or developing prostate cancer include, for example, age, smoking, race, geographic location, family history, and family history (Gann Rev Urol. (2002) 4:S3-S10).

In some embodiments, the subject has prostate cancer or is at risk of having prostate cancer. Whether a subject has prostate cancer or is at risk of having prostate cancer will be evident to one of skill in the art and may be determined by performing an assessment, such as prostate-specific antigen blood test, digital rectal exam, and biopsy. In some embodiments, the subject has one or more symptoms associated with prostate cancer, such as difficult or painful urination, blood in the urine or semen, pressure or pain in the rectum, and pain or stiffness in the lower back, pelvis, hips, and thighs.

The presence of another disease or disorder may predispose a subject to having or being at risk of having prostate cancer. In some embodiments, a subject may be at risk of having prostate cancer if the subject has a chronic inflammatory disorder. For example, an increase in the risk of prostate cancer has be found in individuals with chronic inflammation or an inflammatory disorder, such as prostatitis (see, e.g., Sfanos et al Histopathology (2012) 60(1): 199-215).

Cancers associated with tumor formation may be classified into four stages based on the TNM system of tumor classification. The TNM system of tumor classification or staging involves evaluating the tumor (the “T” score), the extent of the spread to the lymph nodes (the “N” score), and the presence of metastasis (the “M” score). The cancer receives a score based on the cumulative T, N, and M scores. A stage I cancer may have a T score of 1 or 2, a N score of 0, and an M score of 0. A stage II cancer may have a T score of 1 or 2, a N score of 0, and an M score of 0. A stage III cancer may have any T score, a N score of 0, an M score of 0. A stage IV cancer may have any T score, any N score, an M score of 1.

In some embodiments, the subject has stage I cancer. In some embodiments, the subject has stage II cancer. In some embodiments, the subject has stage III cancer. In some embodiments, the subject has stage IV cancer. In some embodiments, the subject has a cancer characterized by chronic inflammation that is stage I cancer. In some embodiments, the subject has a cancer characterized by chronic inflammation that is stage II cancer. In some embodiments, the subject has a cancer characterized by chronic inflammation that is stage III cancer. In some embodiments, the subject has a cancer characterized by chronic inflammation that is stage IV cancer. In some embodiments, the subject has a cancer characterized by expression of GM1 ganglioside that is stage I cancer. In some embodiments, the subject has a cancer characterized by expression of GM1 ganglioside that is stage II cancer. In some embodiments, the subject has a cancer characterized by expression of GM1 ganglioside that is stage III cancer. In some embodiments, the subject has a cancer characterized by expression of GM1 ganglioside that is stage IV cancer. In some embodiments, the subject has stage I colorectal cancer. In some embodiments, the subject has stage II colorectal cancer. In some embodiments, the subject has stage III colorectal cancer. In some embodiments, the subject has stage IV colorectal cancer. In some embodiments, the subject has stage I prostate cancer. In some embodiments, the subject has stage II prostate cancer. In some embodiments, the subject has stage III prostate cancer. In some embodiments, the subject has stage IV prostate cancer.

In some embodiments, the methods include assessing whether a subject has cancer. In some embodiments, the subject may be assessed for cancer prior to administration of the compositions comprising LPS, compositions comprising cholera toxin, or the compositions comprising a combination of LPS and cholera toxin described herein. In some embodiments, if the subject is assessed as having cancer, any of the compositions comprising LPS, compositions comprising cholera toxin, or compositions comprising a combination of LPS and cholera toxin described herein may be administered to the subject.

In some embodiments, the methods include determining whether a subject is predisposed to developing a cancer, such as a characterized by chronic inflammation or by expression of GM1 ganglioside receptors. In some embodiments, determining whether a subject is predisposed to developing a cancer involves assessing the subject for an inflammatory condition associated with cancer. In some embodiments, if the subject is determined to be predisposed to a cancer characterized by chronic inflammation, the subject is administered any of the compositions described herein. In some embodiments, determining whether a subject is predisposed to developing a cancer involves assessing the subject for GM1 ganglioside receptor expression. In some embodiments, if the subject is determined to be predisposed to a cancer characterized by GM1 ganglioside receptor expression, the subject is administered any of the compositions described herein.

In some embodiments, the methods include determining the stage of the cancer, such a cancer characterized by chronic inflammation or expression of GM1 ganglioside. In some embodiments, the stage of the cancer may be determined prior to administration of any of the compositions described herein. In some embodiments, if the cancer is determined to be a particular stage, any of the compositions comprising LPS, compositions comprising cholera toxin, or compositions comprising a combination of LPS and cholera toxin described herein may be administered to the subject. In some embodiments, a composition comprising LPS, compositions comprising cholera toxin, or a composition comprising a combination of LPS and cholera toxin as described herein is administered, if the cancer is determined to be stage I cancer. In some embodiments, a compositions comprising LPS, compositions comprising cholera toxin, or a composition comprising a combination of LPS and cholera toxin as described herein is administered, if the cancer is determined to be stage II cancer. In some embodiments, a composition comprising LPS, compositions comprising cholera toxin, or a composition comprising a combination of LPS and cholera toxin as described herein is administered, if the cancer is determined to be stage III cancer. In some embodiments, a composition comprising LPS, compositions comprising cholera toxin, or a composition comprising a combination of LPS and cholera toxin as described herein is administered, if the cancer is determined to be stage IV cancer.

In some embodiments, the methods include determining the stage of the colorectal cancer or prostate. In some embodiments, the stage of the colorectal cancer or prostate may be determined prior to administration of any of the compositions described herein. In some embodiments, if the colorectal cancer or prostate cancer is determined to be a particular stage, any of the compositions comprising LPS, compositions comprising cholera toxin, or compositions comprising a combination of LPS and cholera toxin described herein may be administered to the subject. In some embodiments, a composition comprising LPS, compositions comprising cholera toxin, or a composition comprising a combination of LPS and cholera toxin as described herein is administered, if the cancer is determined to be stage I colorectal cancer or stage I prostate cancer. In some embodiments, a compositions comprising LPS, compositions comprising cholera toxin, or a composition comprising a combination of LPS and cholera toxin as described herein is administered, if the cancer is determined to be stage II colorectal cancer or stage II prostate cancer. In some embodiments, a composition comprising LPS, compositions comprising cholera toxin, or a composition comprising a combination of LPS and cholera toxin as described herein is administered, if the cancer is determined to be stage III colorectal cancer or stage III prostate cancer. In some embodiments, a composition comprising LPS, compositions comprising cholera toxin, or a composition comprising a combination of LPS and cholera toxin as described herein is administered, if the cancer is determined to be stage IV colorectal cancer or stage IV prostate cancer.

In some embodiments, the subject has undergone or is currently undergoing another therapy to treat the cancer (e.g., cancer characterized by chronic inflammation or expression of GM1 ganglioside). In some embodiments, the subject has undergone a surgical procedure to remove cancer tissue or tissue suspected of being cancer. In some embodiments, the subject has undergone or is currently undergoing an anti-cancer therapy to treat the cancer. In some embodiments, the anti-cancer therapy is an immunotherapy or chemotherapy. In some embodiments, the subject has undergone one or more rounds of chemotherapy to treat the cancer. In some embodiments, the subject has undergone a radiation therapy to treat the cancer.

Examples of chemotherapeutic agents include, without limitation, Methotrexate, Paclitaxel, Brentuximab Vedotin, Doxorubicin, 5-FU (fluorouracil), Everolimus, PEMETREXED, Melphalan, Pamidronate, Anastrozole, Exemestane, Nelarabine, Ofatumumab, Bevacizumab, Belinostat, Tositumomab, Carmustine, Bleomycin, Blinatumomab, Bosutinib, Busulfan, Alemtuzumab, Irinotecan, Vandetanib, Bicalutamide, Lomustine, Daunorubicin, Clofarabine, Cabozantinib, Dactinomycin, Cobimetinib, Ramucirumab, Cytarabine, Cytoxan, Cyclophosphamide, Decitabine, Dexamethasone, Docetaxel, Hydroxyurea, Decarbazine, Leuprolide, epirubicin, oxaliplatin, Asparaginase, Estramustine, Cetuximab, Vismodegib, Asparaginase Erwinia chrysanthemi, Amifostine, Etoposide, Flutamide, Toremifene, Panobinostat, Fulvestrant, Letrozole, Degarelix, Fludarabine, Pralatrexate, floxuridine, Obinutuzumab, Gemcitabine, Afatinib, Imatinib Mesylate, Carmustine wafer, Eribulin, Trastuzumab, Altretamine, Topotecan, Palbociclib, Ponatinib, Idarubicin, Ifosfamide, Ibrutinib, Axitinib, Interferon alfa-2a, Gefitinib, Romidepsin, Ixabepilone, Ruxolitinib, Cabazitaxel, Ado-trastuzumab Emtansine, Pembrolizumab, Carfilzomib, Lenvatinib, Chlorambucil, Sargramostim, Cladribine, Trifluridine and Tipiracil, Olaparib, Mitotane, Vincristine, Procarbazine, Megestrol, Trametinib, Mesna, Strontium-89 Chloride, Mechlorethamine, Mitomycin, Gemtuzumab Ozogamicin, Vinorelbine, Cyclophosphamide, filgrastim, pegfilgrastim, Sorafenib, nilutamide, Pentostatin, Mitoxantrone, Sonidegib, Pegaspargase, Denileukin Diftitox, Nivolumab, Alitretinoin, Carboplatin, Pertuzumab, Cisplatin, Pomalidomide, Prednisone, Aldesleukin, Mercaptopurine, Zoledronic acid, Lenalidomide, Rituximab, Octreotide, Tamoxifen, Dasatinib, Regorafenib, Sunitinib, Peginterferon Alfa-2b, Siltuximab, Omacetaxine, Thioguanine, Dabrafenib, Erlotinib, Bexarotene, Decarbazine, Docetaxel, Temozolomide, Thiotepa, Thalidomide, BCG, Temsirolimus, Bendamustine hydrochloride, Triptorelin, Arsenic trioxide, lapatinib, Dinutuximab, Valrubicin Intravesical, Histrelin, Panitumumab, Vinblastine, Bortezomib, Tretinoin, Azacitidine, Pazopanib, Teniposide, Leucovorin, Crizotinib, Capecitabine, Enzalutamide, Ipilimumab, Trabectedin, Ziv-aflibercept, Streptozocin, Vemurafenib, Ibritumomab, Tiuxetan, Goserelin, Vorinostat, Idelalisib, Ceritinib, and Abiraterone.

Any of the compositions comprising LPS from at least one strain of V. cholerae may be administered to the subject twice or more (e.g., as a triple or quadruple dose). In some embodiments, any of the compositions comprising LPS from at least one strain of V. cholerae may be administered to the subject more than once (e.g., as multiple doses). In some embodiments, the composition comprising LPS from at least one strain of V. cholerae is administered to the subject on at least 2, 3, 4, 5, 6, 7, 8, 9, 10 or more occasions.

Any of the compositions comprising cholera toxin may be administered to the subject twice or more (e.g., as a triple or quadruple dose). In some embodiments, any of the compositions comprising cholera toxin may be administered to the subject more than once (e.g., as multiple doses). In some embodiments, the composition comprising cholera toxin is administered to the subject on at least 2, 3, 4, 5, 6, 7, 8, 9, 10 or more occasions.

Any of compositions comprising a combination of LPS from at least one strain of V. cholerae and cholera toxin may be administered to the subject twice or more (e.g., as a triple or quadruple dose). In some embodiments, any of the compositions comprising a combination of LPS from at least one strain of V. cholerae and cholera toxin may be administered to the subject more than once (e.g., as multiple doses). In some embodiments, the composition comprising a combination of LPS from at least one strain of V. cholerae and cholera toxin is administered to the subject on at least 2, 3, 4, 5, 6, 7, 8, 9, 10 or more occasions.

Each administration of any of the compositions described herein may be in the form of a “dose.” Any administration of the compositions described herein that is administered after the first administration may be referred to as a “booster.” In some embodiments, a composition comprising LPS from at least one strain of V. cholerae is administered to the subject more than twice, e.g. three or four times. In some embodiments, a composition comprising cholera toxin is administered to the subject more than twice, e.g. three or four times. In some embodiments, a composition comprising a combination of LPS from at least one strain of V. cholerae and cholera toxin is administered to the subject more than twice, e.g. three or four times.

In some embodiments, the more than one administration of any of the compositions described herein are administered sequentially to the subject. In some embodiments, a subsequent administration of any of the compositions described herein is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or at least 60 days after the first administration. In some embodiments, a subsequent administration of any of the compositions described herein is administered at least 1 week, 2 weeks, 3 weeks, 4 weeks, 5 weeks, 6 weeks, 7 weeks, 8 weeks, 9 weeks, 10 weeks, 11 weeks, 12 weeks, or longer after the first administration. Determining whether a subject is in need of one or more additional administrations of any of the compositions described herein will be evident to one of ordinary skill in the art.

The compositions described herein may be administered by any route known in the art. Routes of administration include, but are not limited to, oral, intravenous, subcutaneous, parenteral, intratumoral, intramuscular, intranasal, intracranial, sublingual, intratracheal, inhalation, ocular, vaginal, and rectal. In some embodiments, the compositions are orally administered to the subject. In some embodiments, the compositions are parenterally administered to the subject.

Any of the methods or uses described herein may be for the treatment of cancer in a subject. As used herein, the terms “treat” and “treating,” include the administration of a composition comprising LPS from at least one V. cholerae strain, a composition comprising cholera toxin, or a composition comprising a combination of LPS from at least one V. cholerae strain and cholera toxin to a subject to cure, ameliorate, prevent, reduce, or delay the onset of the symptoms, complications, pathologies or biochemical indicia of the disease (e.g., colorectal cancer), alleviating the symptoms or arresting or inhibiting further development of the disease, and/or reduce metastasis of the disease. Treatment may be prophylactic (to prevent or delay the onset of the disease, or to prevent or reduce the manifestation of clinical or subclinical symptoms thereof) or therapeutic suppression or alleviation of symptoms after the manifestation of the disease.

In some embodiments, administration of any of the compositions described herein to a subject reduces the tumor number, burden, and/or metastasis of cancer (e.g., cancer characterized by chronic inflammation or by expression of GM1 ganglioside receptors) in the subject by at least 10%, 15%, 20%, 25%, 30%, 35%, 40%, 45%, 50%, 55%, 60%, 65%, 70%, 75%, 80%, 85%, 90%, 95%, or more as compared to the tumor number, burden, and/or metastasis of the cancer in the subject prior to administration with the composition. In some embodiments, administration of any of the compositions described herein to a subject reduces the tumor number, burden, and/or metastasis of the cancer (e.g., cancer characterized by chronic inflammation or expression of GM1 ganglioside receptors) in the subject by at least 10%, 15%, 20%, 25%, 30%, 35%, 40%, 45%, 50%, 55%, 60%, 65%, 70%, 75%, 80%, 85%, 90%, 95%, or more as compared to the tumor number, burden, and/or metastasis of the cancer in subjects that did not receive the compositions.

In some embodiments, administration of any of the compositions described herein to a subject reduces the severity or incidence of one or more symptoms associated with the cancer (e.g., cancer characterized by chronic inflammation or by expression of GM1 ganglioside receptors) in the subject by at least 10%, 15%, 20%, 25%, 30%, 35%, 40%, 45%, 50%, 55%, 60%, 65%, 70%, 75%, 80%, 85%, 90%, 95%, or more as compared to the severity or incidence of the symptoms in the subject prior to administration with the composition. In some embodiments, administration of any of the compositions described herein to a subject reduces the severity or incidence of one or more symptoms associated with the cancer in the subject by at least 10%, 15%, 20%, 25%, 30%, 35%, 40%, 45%, 50%, 55%, 60%, 65%, 70%, 75%, 80%, 85%, 90%, 95%, or more as compared to the severity or incidence of the symptoms in subjects that did not receive the compositions.

The methods described herein may result in a reduction of the mortality rate or the risk of mortality in the subject being administered the compositions comprising LPS from at least one V. cholerae strain, a composition comprising cholera toxin, or composition comprising a combination of LPS from at least one V. cholerae strain and cholera toxin from at least one V. cholerae strain. As used herein, the term “mortality rate” refers to a measure of the number of deaths (in general or due to a specific cause) in some population, scaled to the size of that population, per unit time, e.g., mortality rate of a group of subjects over 3 years. The mortality rate may be used to assess the efficacy of a particular treatment, e.g. the compositions described herein. In some embodiments, the mortality in subjects who are administered a composition comprising LPS from at least one V. cholerae strain, a composition comprising cholera toxin, or a composition comprising a combination of LPS from at least one V. cholerae strain and cholera toxin (e.g., a treated subject group) is compared to the mortality rate in subjects who did not receive the treatment (i.e., received a placebo control).

Administration of the compositions described herein may result in a reduction in the mortality rate of subjects who receive any of the compositions described herein as compared to subjects who did not receive the compositions. In some embodiments, the mortality rate refers to overall mortality, meaning mortality due to any cause. In some embodiments, the mortality rate refers to mortality associated with or caused by cancer, such as cancer characterized by chronic inflammation or by expression of GM1 ganglioside receptors.

In some embodiments, the mortality rate of subjects that are administered any of the compositions described herein is reduced relative to the mortality rate of subjects were not administered the compositions. In some embodiments, the mortality rate of subjects that are administered any of the compositions described herein is reduced by at least 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or at least 50% compared to subjects that did not receive treatment.

Administration of the compositions described herein may enhance survival of subjects who receive the compositions as compared to subjects who did not receive the compositions. In some embodiments, subjects that receive the compositions described herein have enhanced survival (e.g., survive for a longer period of time following diagnosis of a particular stage of cancer) as compared to subjects that did not receive the compositions described herein. In some embodiments, the subjects that receive the composition described herein survive on average 1 month, 2 months, 3 months, 4 months, 5 months, 6 months, 7 months, 8 months, 9 months, 10 months, 11 months, 12 months, 18 months, 2 years, 3 years, 4 years, 5 years, 6 years, 7 years, 8 years, 9 years, 10 years, or longer than subjects that did not receive the compositions described herein. Preferably, the subjects that receive the compositions described herein survive on average more than 3 years after administration of the compositions of the invention as herein described.

Any of the compositions comprising LPS from at least one V. cholerae strain, compositions comprising cholera toxin, or compositions comprising a combination of LPS from at least one V. cholerae strain and cholera toxin described herein may be administered to a subject with, prior to, or after administration of an acid neutralizing agent. An acid neutralizing agent is any agent that reduces acidity (i.e., increases the pH, neutralizes). Examples of acid neutralizing agents include, without limitation, sodium bicarbonate (sodium hydrogen carbonate), sodium dihydrogen phosphate dihydrate, disodium hydrogen phosphate dihydrate, algeldrate (aluminium hydroxide), magnesium carbonate, calcium carbonate, magnesium hydroxide, and simethicone.

Determining whether subject who administered the compositions comprising LPS from at least one V. cholerae strain, compositions comprising cholera toxin, or compositions comprising a combination of LPS from at least one V. cholerae strain and cholera toxin from at least one V. cholerae strain described herein are also administered an acid neutralizing agent may depend on factors, such as the route of administration and/or components of the compositions. For example, a composition that is administered by an oral route may encounter the acid environment of the stomach. In some embodiments, oral administration of an acid neutralizing agent reduces the acidity (e.g., increases the pH, neutralizes) of the stomach.

In some embodiments, any of the compositions comprising LPS from at least one V. cholerae strains also comprises an acid neutralizing agent. In some embodiments, any of the compositions comprising LPS from at least one V. cholerae strains may be resuspended in an acid neutralizing agent and the composition and acid neutralizing agent are administered simultaneously to the subject. In some embodiments, any of the compositions comprising cholera toxin also comprises an acid neutralizing agent. In some embodiments, any of the compositions comprising cholera toxin may be resuspended in an acid neutralizing agent and the composition and acid neutralizing agent are administered simultaneously to the subject. In some embodiments, any of the compositions comprising a combination of LPS from at least one V. cholerae strains and cholera toxin also comprises an acid neutralizing agent. In some embodiments, any of the compositions comprising a combination of LPS from at least one V. cholerae strains and cholera toxin may be resuspended in an acid neutralizing agent and the composition and acid neutralizing agent are administered simultaneously to the subject.

Any of the compositions comprising LPS from at least one V. cholerae strain, compositions comprising cholera toxin, or compositions comprising a combination of LPS from at least one V. cholerae strain and cholera toxin described herein may be administered to a subject with, prior to, or after administration of one or more additional therapeutic agents. In some embodiments, compositions comprising LPS from at least one V. cholerae strain, compositions comprising cholera toxin, or compositions comprising a combination of LPS from at least one V. cholerae strain and cholera toxin described herein may be administered to a subject with, prior to, or after administration of an immune checkpoint inhibitor. Immune checkpoints are regulatory pathways within the immune system that are involved in maintaining immune homeostasis to minimize cellular damage due to unregulated immune responses. As used herein, the term “immune checkpoint inhibitor” refers to inhibitors of immune checkpoints.

In some embodiments, the immune checkpoint inhibitor is a PD-1 inhibitor, PD-L1 inhibitor, CTLA-4 inhibitor, or a combination thereof. In some embodiments, the immune checkpoint inhibitor is an antibody, such as a monoclonal antibody. In some embodiments, the immune checkpoint inhibitor is a PD-1 inhibitor. In some embodiments, the PD-1 inhibitor is nivolumab or pembrolizumab. In some embodiments, the immune checkpoint inhibitor is a PD-L1 inhibitor. In some embodiments, the PD-L1 inhibitor is atezolizumab, avelumab or durvalumab. In some embodiments, the immune checkpoint inhibitor is a CTLA-4 inhibitor. In some embodiments, the CTLA-4 inhibitor is ipilimumab or tremelimumab.

In some embodiments, the compositions comprising LPS from at least one V. cholerae strain, compositions comprising cholera toxin, or compositions comprising a combination of LPS from at least one V. cholerae strain and cholera toxin described herein may be administered to a subject with, prior to, or after administration of an EGFR inhibitor. EGFR, epidermal growth factor receptor, is a cell surface tyrosine kinase receptor. Interaction with the ligand epidermal growth factor (EGF) results in an intracellular signaling cascade leading to cell proliferation. EGFR inhibitors prevent or disrupt signaling through EGFR. Examples of inhibitors of EGFR small molecule tyrosine kinase inhibitors and monoclonal antibodies, such as necitumumab, erlotinib, afatinib, gefitinib, lapatinib, osimertinib, cetuximab, panitumumab, ABX-EGF, and TheraCIM (h-R3).

In some embodiments, the compositions comprising LPS from at least one V. cholerae strain, compositions comprising cholera toxin, or compositions comprising a combination of LPS from at least one V. cholerae strain and cholera toxin described herein may be administered to a subject with, prior to, or after administration of an VEGF inhibitor. VEGF, vascular endothelial growth factor, is a pro-angiogenic paracrine factor that stimulates cell proliferation. Inhibitors of VEGF prevent or reduce VEGF signaling through VEGFR, the VEGF receptor. In some embodiments, the VEGF inhibitor is a monoclonal antibody. In some embodiments, the VEGF inhibitor is bevacizumab.

Any of the compositions comprising LPS from at least one V. cholerae strain, compositions comprising cholera toxin, or compositions comprising a combination of LPS from at least one V. cholerae strain and cholera toxin described herein may be administered to a subject in a therapeutically effective amount. As used herein, a “therapeutically effective amount” or an “effective amount” of composition is any amount that results in a desired response or outcome in a subject, such as those described herein, including but not limited to preventing or treating cancer, such as cancer characterized by chronic inflammation or by expression of GM1 ganglioside receptors, enhancing survival. In some embodiments, the compositions comprising LPS from at least one V. cholerae strain, compositions comprising cholera toxin, or compositions comprising a combination of LPS from at least one V. cholerae strain and cholera toxin may be formulated for administration in a pharmaceutical composition. The term “pharmaceutical composition” as used herein means a product that results from the mixing or combining of more than one active ingredient and includes both fixed and non-fixed combinations of the active ingredients. The term “fixed combination” means that the active ingredients, e.g. LPS of at least one V. cholerae strain or a combination of LPS of at least one V. cholerae strain and cholera toxin, and a co-agent, are both administered to a patient simultaneously in the form of a single entity or dosage. The term “non-fixed combination” means that the active ingredients, e.g. LPS of at least one V. cholerae strain, compositions comprising cholera toxin, or a combination of LPS of at least one V. cholerae strain and cholera toxin, and a co-agent, are both administered to a patient as separate entities either simultaneously, concurrently or sequentially with no specific time limits, wherein such administration provides therapeutically effective levels of the 2 compounds in the body of the patient.

Pharmaceutical compositions of the disclosure, including vaccines, can be prepared in accordance with methods well known and routinely practiced in the art (see e.g., Remington: The Science and Practice of Pharmacy, Mack Publishing Co. 20th ed. 2000; and Ingredients of Vaccines—Fact Sheet from the Centers for Disease Control and Prevention, e.g., adjuvants, enhancers, preservatives, and stabilizers). Pharmaceutical compositions are preferably manufactured under GMP conditions. The compositions described herein may be formulated into pharmaceutically acceptable dosage forms by conventional methods known to those of skill in the art.

The compositions comprising LPS from at least one strain of V. cholerae, compositions comprising cholera toxin, or compositions comprising a combination of LPS from at least one strain of V. cholerae and cholera toxins are typically administered to subjects as pharmaceutical compositions, which may routinely contain pharmaceutically acceptable concentrations of salt, buffering agents, preservatives, compatible carriers, adjuvants, and optionally other therapeutic ingredients. The nature of the pharmaceutical carrier and other components of the pharmaceutical composition will depend on the mode of administration. The pharmaceutical compositions of the disclosure may be administered by any means and route known to the skilled artisan in carrying out the treatment methods described herein. In some embodiments, the compositions are formulated for oral administration.

Formulations may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. In some embodiments, the compositions are present in a power or lyophilized form for resuspension prior to administration.

The compositions may be formulated for parenteral administration by injection. As used herein, “parenteral” administration includes, without limitation, subcutaneous, intracutaneous, intravenous, intratumoral, intramuscular, intraarticular, intrathecal, or by infusion. Formulations for injection may be presented in unit dosage form, e.g., in ampoules or in multi dose containers, with an added preservative. The compositions may take such forms as suspensions, solutions or emulsions in oily or aqueous vehicles, and may contain formulatory agents such as suspending, stabilizing and/or dispersing agents. Pharmaceutical formulations for parenteral administration include aqueous solutions of the active compositions in water soluble form. Additionally, suspensions of the active compositions may be prepared as appropriate oily injection suspensions. Alternatively, the active compositions may be in powder form for constitution with a suitable vehicle, e.g., sterile pyrogen-free water, before use.

For oral administration, the compositions can be formulated readily by combining the compounds with pharmaceutically acceptable carriers well known in the art. Such carriers enable the compounds of the disclosure to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally, the oral formulations may also be formulated in saline or buffers, e.g., EDTA for neutralizing internal acid conditions, or may be administered without any carriers.

For oral delivery, the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films. A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic powder; for liquid forms, a soft gelatin shell may be used. The shell material of cachets could be thick starch or other edible paper. For pills, lozenges, molded tablets or tablet triturates, moist massing techniques can be used.

Dosage regimens are adjusted to provide the optimum desired response. Dosage levels of the active ingredients in the pharmaceutical compositions of the present disclosure can be varied so as to obtain an amount of the active ingredient which is effective to achieve the desired pharmaceutical response for a particular subject, composition, and mode of administration, without being toxic to the subject. The selected dosage level depends upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the subject being treated, and like factors.

A physician, veterinarian or other trained practitioner, can start doses of compositions comprising LPS of at least one V. cholerae strain, compositions comprising cholera toxin, or compositions comprising a combination of LPS and cholera toxin employed in the pharmaceutical composition at levels lower than that required to achieve the desired therapeutic effect and gradually increase the dosage until the desired effect is achieved. In general, effective doses of the compositions disclosed herein, for the prophylactic and therapeutic treatment of groups of people as described herein vary depending upon many different factors, including means of administration, target site, physiological state of the patient, whether the patient is human or an animal, other medications administered, and whether treatment is prophylactic or therapeutic. Treatment dosages need to be titrated to optimize safety and efficacy.

In some embodiments, the dosage of LPS, cholera toxin, or combination of LPS and cholera toxin in a composition refers to the amount of LPS, cholera toxin, or combination of LPS and cholera toxin that is administered to the subject in the composition. It will be appreciated the amount of LPS, cholera toxin, or combination of LPS and cholera toxin may be presented as the direct weight of LPS, cholera toxin, or combination of LPS and cholera toxin molecules, respectively; the weight of bacterial cells as an indirect amount of LPS, cholera toxin, or combination of LPS and cholera toxin, respectively; or as the quantity of bacterial cells in the composition, referring to the amount of LPS, cholera toxin, or combination of LPS and cholera toxin, respectively, associated with the quantity of bacterial cells. In a preferred embodiment, the dosage of a LPS and/or cholera toxin composition is that of Shanchol®, Euvichol®, Vaxchora® or Dukoral®. In a more preferred embodiment, the dosage of a LPS and/or cholera toxin composition is that of Dukoral®.

In some embodiments, the compositions comprise between about 0.1 μg/mL-10 mg cholera toxin such as e.g. the compositions comprise between about 0.1 μg/mL-10 mg recombinant cholera toxin subunit B per dosage. In some embodiments, the compositions comprise 0.1 μg-5 mg, 0.1 μg-7 mg, 0.1 μg/mL-3 mg, 0.2 μg-4 mg cholera toxin such as e.g. recombinant cholera toxin subunit B per dosage. In some embodiments, the compositions contain about 1 mg cholera toxin such as e.g. recombinant cholera toxin subunit B per dosage.

In some embodiments, the compositions contain between 10⁵ and 10¹⁵ of each V. cholerae strain per dosage. In some embodiments, the compositions contain between 10⁵ and 10¹⁵, between 10⁶ and 10⁴, between 10⁷ and 10¹³, between 10⁸ and 10¹², between 10⁹ and 10¹¹ of each V. cholerae strain per dosage. In some embodiments, the compositions contain between 10¹⁰ and 10¹¹ bacterial cells per dosage. In some embodiments, the compositions contain approximately 3×10¹⁰ cells of each V. cholerae strain per dosage.

In some embodiments, the compositions contain between 10⁵ and 10¹⁵ total V. cholerae cells per dosage. In some embodiments, the compositions contain between 10⁵ and 10¹⁵, between 10⁶ and 10¹⁴, between 10⁷ and 10¹³, between 10⁹ and 10¹², between 10¹⁰ and 10¹² total V. cholerae cells per dosage. In some embodiments, the compositions contain between 10¹¹ and 10¹² bacterial cells per dosage. In some embodiments, the compositions contain approximately 1.25×10¹¹ total V. cholerae cells per dosage.

In some embodiments, the compositions contain between 10⁵ and 10¹⁵ colony-forming units (CFUs) of live-attenuated V. cholerae per dosage. In some embodiments, the compositions contain between 10⁵ and 10¹⁵, between 10⁶ and 10¹⁴, between 10⁷ and 10¹³, between 10⁶ and 10⁷, between 10⁸ and 10⁹ total CFUs of live-attenuated V. cholerae per dosage. In some embodiments, the compositions contain between 10⁸ and 10⁹ bacterial cells per dosage. In some embodiments, the compositions contain approximately 5×10⁸ total CFUs of V. cholerae per dosage.

The invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Unless otherwise defined herein, scientific and technical terms used in connection with the present disclosure shall have the meanings that are commonly understood by those of ordinary skill in the art. Further, unless otherwise required by context, singular terms shall include pluralities and plural terms hall include the singular. The methods and techniques of the present disclosure are generally performed according to conventional methods well-known in the art. Generally, nomenclatures used in connection with, and techniques of biochemistry, enzymology, molecular and cellular biology, microbiology, virology, cell or tissue culture, genetics and protein and nucleic chemistry described herein are those well-known and commonly used in the art. The methods and techniques of the present disclosure are generally performed according to conventional methods well known in the art and as described in various general and more specific references that are cited and discussed throughout the present specification unless otherwise indicated.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove. However, the citation of any reference is not intended to be an admission that the reference is prior art.

### EXAMPLES

### Example 1: Cholera Vaccine Use is Associated with a Reduced Risk of Death in Patients with Colorectal Cancer: A Population-Based Study

As described in Ji et al. (Gastroenterology (2017) S0016-5085(17)36149-8), it was hypothesized that post-diagnostic use of cholera vaccine, which includes both killed whole cells of Vibrio cholera O1 and recombinant cholera toxin B subunit, in CRC patients might be associated with an improved prognosis. Data regarding the risk of CRC mortality and overall mortality among CRC patients who were cholera vaccine users after their cancer diagnosis was retrieved from several national registers in Sweden and compared to matched controls.

### Methods

### Study Population

This retrospective cohort study was approved by the Ethics Committee at Lund University, Sweden. All patients diagnosed with CRC between July 2005 and December 2012 were identified from the Swedish Cancer Register by using the 10th International Classification of Disease (ICD-10) codes C18, C19 and C20.

Tumor characteristics were recorded according to the TNM system, including the size of tumor, nodal status, and presence of metastatic disease, have been recorded in the register since 2002.¹⁵

The CRC patients were cross referenced to the Swedish Prescribed Drug Registry to retrieve information about cholera vaccine use. The Anatomical Therapeutic Chemical (ATC) code J07AE O1 was used to identify individuals who had been prescribed and administered the cholera vaccine, Dukoral®. Dukoral® contains inactivated Vibrio cholera O1 and recombinant cholera toxin B subunit (not the holotoxin). In total, 175 CRC patients were identified who had received the cholera vaccine after their cancer diagnosis. To mitigate the selection bias of using cholera vaccine, multiple logistic regression analyses were performed for the whole CRC cohort including all the covariates listed in Table 1 and controlled for the remaining imbalance by using nearest neighbor propensity scores matching. The propensity score is the probability of treatment assignment conditional on observed baseline characteristics. A 1-to-3 match was performed so the two groups of patients had the same/similar propensity score. After being matched by propensity scores, the propensity-matched pairs (study cohorts versus matched controls) did not have any significant difference in the demographic and clinical characteristics listed in Table 1. In total, 525 matched controls were included in the study.

The CRC patients were further cross-referenced to Statistics Sweden's Total Population Register and to the Population Housing Census to obtain information on individual-level characteristics such as birth year, years of education, to the Cause of Death Register to identify date of death as well as the cause of death, and to the Emigration Registry to identify date of emigration. All linkages were performed using individual national identification numbers, which were replaced with serial numbers in order to preserve anonymity.

### Study Outcome

From the Cause of Death Register, CRC patients who died between July 2005 and December 2015 were identified, thus ensuring that the entire study cohort had at least three years of follow-up. The primary outcome was death due to CRC (ICD codes: C18, C19 and C20). The secondary outcome was death due to all the causes (ICD codes: A00-Z99).

### Statistical Analysis

Cox regression was used to calculate hazard ratios (HRs) and 95% confidence intervals (CIs) for CRC mortality and all-cause mortality associated with post-diagnostic use of cholera vaccine.

Potential confounding was controlled by the selected clinical and demographic characteristics. The following characteristics were included in the model: age at diagnosis of CRC (modeled as a continuous variable); year at diagnosis of CRC (modeled as a continuous variable to account for follow-up time); gender (male and female), stage of CRC (stage I to IV); country of birth (Sweden, European countries, and others); highest educational level (<9, 9-11, or ≥12 years).

Individuals were censored (i.e., treated them as no longer under observation or at risk of the study outcome) at the time of death from any cause, at the end of the follow-up period (Dec. 31, 2015) or at the time of emigration, whichever came first. To account for immortal time bias, the study population was followed from the date of cholera vaccination to the earliest of death, emigration, or the end of the study period (Dec. 31, 2015).¹⁸ A comparable time at the start of follow-up was adopted for the matched controls. The proportional hazards assumption was tested using cumulative martingale residuals and the Kolmogorov-based supremum test. Data are accurate to two decimal places. All analyses were performed using SAS version 9.2 (SAS Institute, Cary, N.C., USA).

### Sensitivity Analyses

Confounding by indication (indication bias) is a common confounder in observational pharmacoepidemiologic studies. It arises from the fact that individuals who are prescribed a medication are inherently different from those who do not take the drug because they are given a medication for a reason. The exact reason why the CRC patients received the cholera vaccinations is not known. However, it is hypothesized that those patients who received a cholera vaccination may travel outside Sweden. To control for this indication bias, the mortality in CRC patients who had used antimalarial medications after their diagnosis was examined, based on the fact that most Swedes that received antimalarial medications might have travelled abroad (data not shown).

### Results

Between July 2005 and December 2012 (Table 1), a total of 175 patients in Sweden were diagnosed with CRC and were prescribed cholera vaccine after their cancer diagnosis. In addition, 525 CRC patients were identified as their propensity score matched controls. The median age at diagnosis of CRC was 62 years for the study population and the matched controls. Most of the CRC patients were born in Sweden (92%), whereas immigrants accounted for approximately 8%. Among those who received the cholera vaccine, 28.0% had stage I CRC at diagnosis, 34.3% had stage II CRC at diagnosis, 34.3% had stage III CRC at diagnosis, and 3.4% had stage IV CRC at diagnosis

After a mean of 7.0 years and accumulated 1225 person-years of follow-up, 15 CRC patients who had received the cholera vaccine had died due to the illness. This yielded an incidence rate of CRC mortality of 12.2 per 1000 person-years (Table 2), whereas the incidence rate of CRC mortality was 19.7 for CRC patients that had not received the cholera vaccine. Administration of the cholera vaccine after CRC diagnosis was associated with a decreased HR of 0.53 (95% CI 0.29-0.99). The association between post diagnostic use of cholera vaccine and CRC mortality was further stratified by age at diagnosis, gender, and stage at diagnosis. Risk reduction associated with cholera vaccine was largely consistent irrespective of these stratification factors. However, stratified analyses were not statistically significant because of the limited number of cases.

| | |
|---|---|
| Study | Matched |
| cohorts | controls |

In Table 3, the risk of overall mortality associated with cholera vaccine use was examined. The overall mortality was 21.2 per 1000 person-years in CRC patients who had received the cholera vaccine, whereas the overall mortality rate was 33.6 for CRC patients who had not received the cholera vaccine. Administration of the cholera vaccine after CRC diagnosis was associated with a decreased overall mortality (HR=0.59, 95% CI 0.37-0.94). Stratification of the observed association by age at diagnosis, gender, and stage at diagnosis showed that the decrease was largely consistent. However, none of these stratified data showed a significant decrease.

To control the confounding by indication, the mortality in CRC patients who had used antimalarial medications after their diagnosis was further examined (data not shown). The CRC patients that received antimalarial medications showed an increased CRC and overall higher mortality rate as compared to matched controls. The analyses were also stratified by histology of CRC (data not shown). More than 90% of these CRC patients were diagnosed with adenocarcinoma and showed a similar HR as compared to Table 2 and 3.

TABLE 1
Baseline demographic and clinical
characteristics of the study cohorts and
propensity score matched controls

| | No. of | | No. of | | |
|---|---|---|---|---|---|
| Characteristic | patients | % | patients | % | p value |
| Overall | 175 | 100.0 | 525 | 100.0 | |
| Age at diagnosis | | | | | |
| <50 | 24 | 13.7 | 58 | 11.0 | 0.55 |
| 50-59 | 45 | 25.7 | 150 | 28.6 | |
| 60-69 | 81 | 46.3 | 227 | 43.2 | |
| 70+ | 25 | 14.3 | 90 | 17.1 | |
| Median age, years | 62 | 35.4 | 62 | 11.8 | |
| Gender | | | | | |
| Male | 85 | 48.6 | 275 | 52.4 | 0.39 |
| Female | 90 | 51.4 | 250 | 47.6 | |
| Year at diagnosis, | 2008 | | 2008 | | 0.54 |
| median | | | | | |
| Highest education, | | | | | |
| years | | | | | |
| 1-9 | 25 | 14.3 | 56 | 10.7 | 0.43 |
| 10-11 | 72 | 41.1 | 231 | 44.0 | |
| 12+ | 78 | 44.6 | 238 | 44.0 | |
| Birth country | | | | | |
| Sweden | 161 | 92.0 | 488 | 93.0 | 0.82 |
| European countries | 10 | 5.7 | 24 | 4.6 | |
| Others | 4 | 2.3 | 13 | 2.5 | |
| Stage at diagnosis | | | | | |
| Stage 1 | 49 | 28.0 | 132 | 25.1 | 0.88 |
| Stage 2 | 60 | 34.3 | 182 | 34.7 | |
| Stage 3 | 60 | 34.3 | 191 | 36.4 | |
| Stage 4 | 6 | 3.4 | 20 | 3.8 | |

TABLE 2
Association between post diagnostic use of cholera
vaccine and the risk of colorectal cancer mortality

| | | No. of | | | | | | |
|---|---|---|---|---|---|---|---|---|
| Charac- | No. | person- | No. of | | | | | P |
| teristic | patients | years | deaths | IR | HR | 95% CI | | value |
| Post | | | | | | | | |
| diagnostic | | | | | | | | |
| use of | | | | | | | | |
| cholera | | | | | | | | |
| vaccine | | | | | | | | |
| No | 525 | 3363 | 66 | 19.7 | | Reference | | |
| Yes | 175 | 1225 | 15 | 12.2 | 0.53 | 0.29 | 0.99 | 0.04 |
| Age at | | | | | | | | |
| diagnosis | | | | | | | | |
| <60 | 69 | 494 | 5 | 10.1 | 0.69 | 0.19 | 2.47 | 0.56 |
| >=60 | 106 | 730 | 10 | 13.7 | 0.53 | 0.24 | 1.19 | 0.12 |
| Gender | | | | | | | | |
| Male | 85 | 669 | 6 | 9.0 | 0.53 | 0.19 | 1.43 | 0.20 |
| Female | 90 | 642 | 9 | 14.0 | 0.54 | 0.22 | 1.33 | 0.17 |
| Stage at | | | | | | | | |
| diagnosis | | | | | | | | |
| Stages 1 | 109 | 791 | 5 | 6.3 | 0.53 | 0.17 | 1.59 | 0.25 |
| and 2 | | | | | | | | |
| Stages 3 | 66 | 433 | 10 | 23.1 | 0.51 | 0.22 | 1.19 | 0.11 |
| and 4 | | | | | | | | |

TABLE 3
Association between post diagnostic use of cholera
vaccine and the risk of all-cause mortality

| | | No. of | | | | | | |
|---|---|---|---|---|---|---|---|---|
| Charac- | No. | person- | No. of | | | | | P |
| teristic | patients | years | deaths | IR | HR | 95% CI | | value |
| Post | | | | | | | | |
| diagnostic | | | | | | | | |
| use of | | | | | | | | |
| cholera | | | | | | | | |
| vaccine | | | | | | | | |
| No | 525 | 3363 | 113 | 33.6 | | Reference | | |
| Yes | 175 | 1225 | 26 | 21.2 | 0.59 | 0.37 | 0.94 | 0.02 |
| Age at | | | | | | | | |
| diagnosis | | | | | | | | |
| <60 | 69 | 494 | 5 | 10.1 | 0.53 | 0.16 | 1.78 | 0.30 |
| >=60 | 106 | 730 | 21 | 28.8 | 0.61 | 0.35 | 1.05 | 0.07 |
| Gender | | | | | | | | |
| Male | 85 | 669 | 14 | 20.9 | 0.58 | 0.29 | 1.13 | 0.10 |
| Female | 90 | 642 | 12 | 18.7 | 0.57 | 0.27 | 1.22 | 0.14 |
| Stage at | | | | | | | | |
| diagnosis | | | | | | | | |
| Stages 1 | 109 | 791 | 10 | 12.6 | 0.51 | 0.24 | 1.08 | 0.07 |
| and 2 | | | | | | | | |
| Stages 3 | 66 | 433 | 16 | 36.9 | 0.74 | 0.39 | 1.40 | 0.30 |
| and 4 | | | | | | | | |

### Discussion

In this retrospective cohort study, a total of 175 CRC patients were identified who had been prescribed a cholera vaccine after their cancer diagnosis. As compared to matched controls, CRC patients who received the cholera vaccine after CRC diagnosis had a 47% decrease in CRC mortality and a 41% decrease in overall mortality as compared to CRC patients who did not receive the cholera vaccine. In addition, the decreased risk of mortality was found to be largely consistent irrespective of age at diagnosis, gender, and stage at diagnosis of CRC. These findings suggest that cholera vaccine use may protect against mortality in patients with CRC.

This population-based study has a number of strengths and some limitations. An important strength is that it is a nationwide population-based study. However, cholera vaccine use is relatively uncommon in Sweden. As a consequence, only 175 CRC patients were identified who met the study criteria at a national level. This calls for further international collaborations to confirm the observed association and to identify which groups of patients might benefit most from using cholera vaccine. All the data used in this study were retrieved from nationwide Swedish registers that are of high quality and are considered to be accurate. The prospective study design and the completeness of the follow-up of patients are other major advantages of the present study. Some potential confounding factors could be identified by linking several national registers in Sweden, such as education level and countries of birth, which have been shown to be associated with CRC outcomes.^{19,20} In addition, tumor stage could be adjusted for, which is the most significant factor that affects CRC outcome. One limitation of this study, which may have partly confounded the conclusion, is the lack of information on some individual-level risk factors, such as medical treatments, smoking status, and dietary factors. However, Sweden is well-known for its widespread availability of healthcare for all citizens at a minimal cost; discrepancy in medical treatment of CRC is thus relatively uncommon in Sweden. In addition, education level was adjusted for in the regression model, which may partly minimize the confounding by smoking and dietary factors.^{21,22}

Another limitation is the lack of certainty of medical adherence, which means that it is not certain whether patients complied with the treatment regimen. The rate of non-compliance could be low as the reported side effects are relatively rare²³ and only two doses of cholera vaccines are needed to induce the protection. Uncertainty of medical adherence may cause the possibility of exposure misclassifications. However, such exposure misclassifications might dilute the observed risk toward the null and underestimate the observed risk reduction in this study. Furthermore, confounding by indication could not be totally controlled in this study. However, this issue has been explored by examining the mortality rate in CRC patients who received antimalarial medications based on the assumption that patients that used either cholera vaccine or antimalarial medications might travel abroad. These data suggested that confounding by indication may not contribute to the observed association.

In summary, the results of this population-based study indicated that administration of a cholera vaccine after CRC diagnosis is associated with a decreased risk of cancer-related mortality and all-cause mortality. Risk reduction was largely consistent irrespective of age at diagnosis, gender, and stage at diagnosis.

### REFERENCES

1. Arnold M, Sierra M S, Laversanne M, et al. Global patterns and trends in colorectal cancer incidence and mortality. Gut 2017; 66(4):683-91. doi: 10.1136/gutjnl-2015-310912 [published Online First: 2016/01/29]
2. American Cancer Society. Cancer Facts & Figures 2017. Atlanta, Ga.: American Cancer Society; 2017.
3. Jemal A, Clegg L X, Ward E, et al. Annual report to the nation on the status of cancer, 1975-2001, with a special feature regarding survival. Cancer 2004; 101(1):3-27. doi: 10.1002/cncr.20288 [published Online First: 2004/06/29]
4. Ait Ouakrim D, Pizot C, Boniol M, et al. Trends in colorectal cancer mortality in Europe: retrospective analysis of the WHO mortality database. Bmj 2015; 351:h4970. doi: 10.1136/bmj.h4970 [published Online First: 2015/10/08]
5. Oikonomopoulou K, Brinc D, Kyriacou K, et al. Infection and cancer: revaluation of the hygiene hypothesis. Clinical cancer research: an official journal of the American Association for Cancer Research 2013; 19(11):2834-41. doi: 10.1158/1078-0432.ccr-12-3661 [published Online First: 2013/03/29]
6. Baxter N T, Zackular J P, Chen G Y, et al. Structure of the gut microbiome following colonization with human feces determines colonic tumor burden. Microbiome 2014; 2:20. doi: 10.1186/2049-2618-2-20 [published Online First: 2014/06/27]
7. Zackular J P, Baxter N T, Iverson K D, et al. The gut microbiome modulates colon tumorigenesis. mBio 2013; 4(6):e00692-13. doi: 10.1128/mBio.00692-13 [published Online First: 2013/11/07]
8. Zackular J P, Baxter N T, Chen G Y, et al. Manipulation of the Gut Microbiota Reveals Role in Colon Tumorigenesis. mSphere 2016; 1(1) doi: 10.1128/mSphere.00001-15 [published Online First: 2016/06/16]
9. Belcheva A, Irrazabal T, Robertson S J, et al. Gut microbial metabolism drives transformation of MSH2-deficient colon epithelial cells. Cell 2014; 158(2):288-99. doi: 10.1016/j.cell.2014.04.051 [published Online First: 2014/07/19]
10. Doulberis M, Angelopoulou K, Kaldrymidou E, et al. Cholera-toxin suppresses carcinogenesis in a mouse model of inflammation-driven sporadic colon cancer. Carcinogenesis 2015; 36(2):280-90. doi: 10.1093/carcin/bgu325 [published Online First: 2015/01/01]
11. Poutahidis T, Angelopoulou K, Erdman S E. Old enemies meet new friends for colon cancer prevention. Oncoimmunology 2015; 4(10):e1027474. doi: 10.1080/2162402x.2015.1027474 [published Online First: 2015/10/10]
12. Baldauf K J, Royal J M, Kouokam J C, et al. Oral administration of a recombinant cholera toxin B subunit promotes mucosal healing in the colon. Mucosal immunology 2016 doi: 10.1038/mi.2016.95 [published Online First: 2016/11/03]
13. O'Keefe S J. Diet, microorganisms and their metabolites, and colon cancer. Nature reviews Gastroenterology & hepatology 2016; 13(12):691-706. doi: 10.1038/nrgastro.2016.165 [published Online First: 2016/11/17]
14. Ji J, Sundquist K, Sundquist J, et al. Comparability of cancer identification among Death Registry, Cancer Registry and Hospital Discharge Registry. International journal of cancer Journal international du cancer 2012; 131(9):2085-93. doi: 10.1002/ijc.27462 [published Online First: 2012/02/07]
15. Liu X, Ji J, Sundquist K, et al. The impact of type 2 diabetes mellitus on cancer-specific survival: a follow-up study in Sweden. Cancer 2012; 118(5):1353-61. doi: 10.1002/cncr.26420 [published Online First: 2011/07/30]
16. Hemminki K, Santi I, Weires M, et al. Tumor location and patient characteristics of colon and rectal adenocarcinomas in relation to survival and TNM classes. BMC cancer 2010; 10:688. doi: 10.1186/1471-2407-10-688 [published Online First: 2010/12/24]
17. Hamilton S, Aaltonen L. Tumours of the digestive system. Lyon: IARC; 2000.
18. Suissa S. Immortal time bias in pharmaco-epidemiology. American journal of epidemiology 2008; 167(4):492-9. doi: 10.1093/aje/kwm324 [published Online First: 2007/12/07]
19. Cavalli-Bjorkman N, Lambe M, Eaker S, et al. Differences according to educational level in the management and survival of colorectal cancer in Sweden. European journal of cancer 2011; 47(9):1398-406. doi: 10.1016/j.ejca.2010.12.013 [published Online First: 2011/01/18]
20. Akinyemiju T, Meng Q, Vin-Raviv N. Race/ethnicity and socio-economic differences in colorectal cancer surgery outcomes: analysis of the nationwide inpatient sample. BMC cancer 2016; 16:715. doi: 10.1186/s12885-016-2738-7 [published Online First: 2016/09/07]
21. Eek F, Ostergren P O, Diderichsen F, et al. Differences in socioeconomic and gender inequalities in tobacco smoking in Denmark and Sweden; a cross sectional comparison of the equity effect of different public health policies. BMC public health 2010; 10:9. doi: 10.1186/1471-2458-10-9 [published Online First: 2010/01/13]
22. Ax E, Warensjo Lemming E, Becker W, et al. Dietary patterns in Swedish adults; results from a national dietary survey. The British journal of nutrition 2016; 115(1):95-104. doi: 10.1017/s0007114515004110 [published Online First: 2015/10/23]
23. Lopez-Gigosos R, Garcia-Fortea P, Reina-Dona E, et al. Effectiveness in prevention of travellers' diarrhoea by an oral cholera vaccine WC/rBS. Travel medicine and infectious disease 2007; 5(6):380-4. doi: 10.1016/j.tmaid.2007.06.001 [published Online First: 2007/11/07]

### Example 2: Mouse Model of Inflammation-Driven Colorectal Cancer

A mouse model of inflammation-driven colorectal cancer is established as described, for example, in Doulberis et al. Carcinogenesis (2015) 36(2): 280-290 and Poutahidis et al. Oncolmmunology (2015) 4(10), e1027474; or Parang et al. Methods Mol. Biol. (2016) 1422: 297-307. Briefly, mice receive a single intraperitoneal injection of azoxymethane (AOM) (10 mg/mL) to initiate carcinogenesis. Mice are randomly assigned to one of the following treatment groups:

Group 1: control.

Group 2: V. cholerae only (containing heat inactivated V. cholerae O1 Inaba, classical biotype; formalin inactivated V. cholerae O1 Inaba, El Tor biotype; heat inactivated V. cholerae O1 Ogawa, classical biotype; and formalin inactivated V. cholerae O1 Ogawa, classical biotype.

Group 3: recombinant cholera toxin B subunit only.

Group 4: LPS only.

Group 5: recombinant cholera toxin B subunit and LPS.

Group 6: combination of V. cholerae (containing heat inactivated V. cholerae O1 Inaba, classical biotype; formalin inactivated V. cholerae O1 Inaba, El Tor biotype; heat inactivated V. cholerae O1 Ogawa, classical biotype; and formalin inactivated V. cholerae O1 Ogawa, classical biotype and recombinant cholera toxin B subunit (Dukoral®).

Group 7: lyophilized V. cholerae CVD 103-HgR (i.e., Vaxchora®).

The mice are subjected to three cycles of 1% dextran sodium sulfate in the drinking water for one week and regular drinking water for one week.

At various time points, the mice are sacrificed and the colons removed for analysis. Tissue sections, lymph nodes, and/or cellular samples of the colons may be subjected to gene expression analyses, histopathology, immunohistochemistry methods to evaluate the extent of inflammation and tumor development and progression in each of the groups of mice.

### Example 3: Association Between Post-Diagnostic Use of Cholera Vaccine and Risk of Death in Prostate Cancer Patients

We hypothesized that cholera toxin might have multiple functions regarding the ability to regulate the immune system. However, it was unknown whether subsequent administration of cholera vaccine, which includes both killed whole cells of Vibrio cholerae O1 and recombinant cholera toxin B subunit, would affect the mortality rate of patients with prostate cancer. Data was retrieved from several national registers in Sweden regarding the mortality of patients diagnosed with prostate cancer. The mortality of patients diagnosed with prostate cancer who were administered cholera vaccine was compared to the mortality of prostate cancer patients who did not receive cholera vaccine and to matched controls.

### Methods

### Study Population:

The Ethics Committee at Lund University approved this retrospective cohort study. This study was performed by combining several nation-wide registers.^{58,59} All patients with prostate cancer, who were diagnosed between July 2005 and December 2014, were identified from the Swedish Cancer Registry by using the 10^{th} International Classification of Disease (ICD-10) code C61. The TNM staging system, including the size of the tumor (T), nodal status (N), and the presence of metastatic disease (M), has been incorporated into the Swedish Cancer Registry since 2002. The stage at diagnosis of prostate cancer was determined by combining T, N, and M categories, which ranged from stage I (the least advanced) to stage IV (the most advanced). The stage was defined as follows: stage I (T1 or T2a N0 M0), stage II (T2b or T2c N0 M0), stage III (T3 N0 M0), and stage IV (T4 N1 M1).⁶⁰ Patients with prostate cancer were further linked to the Swedish Prescribed Drug Register to retrieve information about subsequent cholera vaccine use. The Swedish Prescribed Drug Register was established in July 2005 by the National Board of Health and Welfare and has almost 100% complete information about age and sex, as well as information regarding drug utilization and expenditures for all prescribed drugs in the Swedish population. The Anatomical Therapeutic Chemical (ATC) classification system was adopted in the register and used to classify all the drugs. Individuals who had been prescribed and dispensed with cholera vaccine were identified by using the ATC code J07AE01. In Sweden, cholera vaccine is composed of inactivated Vibrio cholera O1 and the recombinant CTB subunit, which is sold under the product name Dukoral®. This study included a total of 841 patients who used cholera vaccine and 89,142 patients who did not use cholera vaccine. To mitigate the possibility that patients using cholera vaccine might be healthier and be associated with a high socioeconomic status, multiple logistic regression analyses were conducted for the whole cohort and adjusted all the covariates listed in Table 4. The remaining imbalance was controlled by matching using the nearest-neighbor propensity scores, which is the probability of receiving cholera vaccination conditional on the observed baseline characteristics. A 1-to-3 match was performed so that the two groups of patients had the same/similar propensity score (Table 6). After being matched by propensity scores, the propensity-matched pairs (patients receiving cholera vaccination vs. patients without vaccination) did not have any significant difference in the demographic and clinical characteristics listed in Table 4.

Patients with prostate cancer were further combined with other registers, such as with the Total Population Register and with the Population Housing Census to obtain information on individual-level characteristics. Patients who died during the study period were identified from the Cause of Death Register. All linkages were performed using individual national identification numbers, which were replaced with serial numbers in order to preserve anonymity. Informed consent was not necessary in this study, as we used register-based data and replaced individuals' identification numbers to ensure anonymity.

### Study Outcome:

From the Cause of Death Register, all patients with prostate cancer who died between July 2005 and December 2015 were identified. The primary outcome was death due to prostate cancer as the primary cause of death (ICD code: C61). The secondary outcome was death due to all the causes (ICD codes: A00-Z99).

### Statistical Analyses:

To account for immortal time bias, time-dependent Cox regression was used to calculate HRs and 95% Cis for prostate cancer mortality and all-cause mortality. Use of cholera vaccine after the diagnosis of prostate cancer was treated as a time-dependent variable in the model, thus allowing patients who moved from a follow-up period of non-exposure (from diagnosis of prostate cancer to the administration of cholera vaccine) to a period of exposure (being vaccinated with cholera vaccine and thereafter for the remainder of follow-up). Several clinical and demographic factors listed in Table 5, which were associated with mortality in patients with prostate cancer, were included in the regression model to account for their potential confounding effects. The model included patient age at diagnosis of prostate cancer, year at diagnosis of prostate cancer, individual disposable income (lowest, middle-low, middle-high, and highest), region at diagnosis (big cities, southern and northern Sweden), stage of prostate cancer (stages 1, 2, 3, and 4) country of birth (Sweden, European countries, and others), the highest educational level (1-9, 10-11, and 12+), and comorbidities (yes or no).

Individuals were censored (i.e., treated them as no longer under observation or at a risk of the study outcome) at the time of death from any cause, at the end of the follow-up period (Dec. 31, 2015), or at the time of emigration, whichever came first. The proportional hazards assumption was tested using cumulative martingale residuals, as shown in Table 7. If the variables did not meet the assumption, we used a stratification model and they were treated as stratification variables. All analyses were performed using SAS version 9.2 (SAS Institute, Cary, N.C., USA).

### Sensitivity Analyses:

Several sensitivity analyses were performed to reduce the possibility of chance findings. First, exposure to cholera vaccine was lagged one year after the administration of cholera vaccine; given that short duration of exposures are unlikely to be associated with the mortality of outcomes.⁶¹ Second, the effect of competing risks as a result of death from other causes were evaluated by using the subdistribution hazards model proposed by Fine and Gray.⁶² Third, all prostate cancer cases diagnosed by PSA screening (defining screening-detected prostate cancer by using the clinical stage of T1c) were removed to exclude its potential effect on the observed associations, because patients with screening-detected prostate cancer might be associated with health behaviors leading to healthy user bias. Fourth, confounding by indication (indication bias) was controlled for by examining the mortality in patients with prostate cancer who had used antimalarial medications, given the probability that most Swedes who received antimalarial medications or cholera vaccine might have traveled abroad. Fifth, the HRs were calculated using patients without vaccination and matched them by a propensity score as the reference (Table 6).

### Results

Characteristics Inpatients with Prostate Cancer

From the Swedish Cancer Registry and Prescribed Drug Register, 841 patients were identified who were prescribed with cholera vaccine after their prostate cancer diagnosis between July 2005 and December 2014 (Table 4). The mean internal time from the diagnosis of prostate cancer to vaccination was 27.1 months, and 58.9 months from vaccination to the end of follow-up. The median age at diagnosis was 64 years. Compared to patients without cholera vaccination, patients who were given a cholera vaccination were diagnosed at a younger age and at an earlier stage, associated with a higher education, a higher disposable income, living in big cities, and with a lower rate of comorbidities.

TABLE 4
Baseline demographic and clinical
characteristics of patients with prostate cancer,
stratified by post-diagnostic use of cholera vaccine

| | Post- | | | | | |
|---|---|---|---|---|---|---|
| | diagnostic | | | | | |
| | uses | | | Without | | |
| | of cholera | | | cholera | | |
| | vaccine | | | vaccine | | |
| | No. of | | | No. of | | |
| Characteristic | patients | | % | patients | % | p value |
| Overall | 841 | | 100.0 | 89,142 | 100.0 | |
| Time interval, | | | | | | |
| mean (SD) | | | | | | |
| Diagnosis to vaccine | 27.1 | (21.6) | | | | |
| Vaccine to end | 58.9 | (26.1) | | | | |
| of follow-up | | | | | | |

| | | | | | |
|---|---|---|---|---|---|
| Age at diagnosis | | | | | |
| <60 | 225 | 26.8 | 12,028 | 13.5 | <0.0001 |
| 60-64 | 220 | 26.2 | 15,972 | 17.9 | |
| 65-69 | 225 | 30.4 | 21,000 | 23.6 | |
| 70+ | 141 | 16.8 | 40,142 | 45.0 | |
| Median age, years | 64 | | 68 | | |
| Year at diagnosis | | | | | |
| 2005-2009 | 628 | 74.8 | 36,884 | 41.4 | <0.0001 |
| 2010-2014 | 213 | 25.4 | 52,258 | 58.6 | |
| Median | 2008 | | | 2010 | |
| Highest | | | | | |
| education, years | | | | | |
| 1-9 | 154 | 18.3 | 30,041 | 33.7 | <0.0001 |
| 10-11 | 302 | 36.0 | 33,613 | 37.7 | |
| 12+ | 385 | 45.8 | 25,488 | 28.6 | |
| Birth country | | | | | |
| Sweden | 795 | 94.6 | 81,179 | 91.1 | 0.002 |
| European countries | 38 | 4.5 | 6,343 | 7.1 | |
| Others | 8 | 1.0 | 1,620 | 1.8 | |
| Income | | | | | |
| Lowest | 71 | 8.5 | 22,870 | 25.7 | <0.0001 |
| Middle-low | 129 | 15.4 | 22,228 | 24.9 | |
| Middle-high | 213 | 25.4 | 22,129 | 24.8 | |
| Highest | 428 | 51.0 | 21,195 | 24.6 | |
| Region | | | | | |
| Big cities | 450 | 53.6 | 42,184 | 47.3 | 0.001 |
| Southern Sweden | 254 | 30.2 | 29,232 | 32.8 | |
| Northern Sweden | 137 | 16.3 | 17,726 | 19.9 | |
| Stage at diagnosis | | | | | |
| Stage 1 | 538 | 64.0 | 43,244 | 48.5 | <0.0001 |
| Stage 2 | 200 | 23.8 | 25,526 | 28.6 | |
| Stage 3 | 68 | 8.1 | 11,759 | 13.2 | |
| Stage 4 | 35 | 4.2 | 8,613 | 9.7 | |
| Chronic ischemic | | | | | |
| heart disease | | | | | |
| No | 750 | 89.3 | 73,991 | 83.0 | <0.0001 |
| Yes | 91 | 10.8 | 15,151 | 17.0 | |
| Diabetes | | | | | |
| No | 794 | 94.5 | 81,375 | 91.3 | 0.001 |
| Yes | 47 | 5.6 | 7,767 | 8.7 | |
| COPD | | | | | |
| No | 819 | 97.5 | 84,226 | 94.5 | 0.001 |
| Yes | 22 | 2.6 | 4,916 | 5.5 | |
| Hypertension | | | | | |
| No | 635 | 75.6 | 63,960 | 71.8 | 0.020 |
| Yes | 206 | 24.5 | 25,182 | 28.2 | |

### Risk of Death Due to Prostate Cancer

The mortality rate in patients with prostate cancer was associated with a range of clinical and demographic factors (Table 5). In Table 6, the adjusted HRs of prostate cancer mortality among patients with post-diagnostic use of cholera vaccine are shown, as compared to patients who did not use cholera vaccine, which included all the potential confounding factors listed in Table 5. After a mean age of 7.2 years and accumulated 6,033 person-years of follow-up, 29 of them had died due to prostate cancer, giving a mortality rate of 4.8 per 1000 person-years, whereas the rate was 22.5 for those patients who did not use cholera vaccine. Use of cholera vaccine was associated with a decreased mortality rate, as compared to patients without cholera vaccination, with a crude HR of 0.33 (95% CI 0.23-0.47) and an adjusted HR of 0.57 (95% CI 0.40-0.82) after adjusting for a range of demographic and clinical factors. The results were similar when using patients who were not vaccinated and matched by a propensity score as the reference (Table 7). The observed association was stratified by patient age at diagnosis, clinical stage, and disposable income. Reduced mortality associated with cholera vaccine was largely inconsistent, irrespective of these stratification factors. Although the point estimate was nearly identical among those patients diagnosed at a young age (<65-years old) and at an early stage (stages 1 and 2), the statistical significance was attenuated due to the limited number of outcomes. We also examined the risk of death due to prostate cancer among patients who received cholera vaccination before the diagnosis of cancer. The HR was 0.76 (95% CI 0.6-0.96) for those who received a vaccination as compared to the non-exposed cases. We further explored the actual HR as a function of the follow-up time (FIG. 2). The figure indicates that the decreased HR was stronger after the vaccination of cholera vaccine, and it gradually became weaker after approximately 15 months.

TABLE 5
Association of prostate cancer mortality
with clinical and demographic characteristics

| | | No. of | | | | | | |
|---|---|---|---|---|---|---|---|---|
| | No. | person- | No. of | | | | | P |
| Characteristic | patients | years | deaths | IR | HR | 95% CI | | value |
| Age at Diagnosis | | | | | | | | |
| <60 | 12253 | 59981 | 514 | 8.6 | | Reference | | |
| 60-64 | 16192 | 80092 | 762 | 9.5 | 1.11 | 0.99 | 1.24 | 0.067 |
| 65-69 | 21255 | 93194 | 1183 | 12.7 | 1.48 | 1.33 | 1.64 | <0.001 |
| 70+ | 40283 | 155799 | 6192 | 39.7 | 4.62 | 4.22 | 5.05 | <0.001 |
| Year at Diagnosis | | | | | | | | |
| 2005-2009 | 37512 | 248699 | 5741 | 23.1 | | Reference | | |
| 2010-2014 | 52471 | 140368 | 2910 | 20.7 | 0.81 | 0.77 | 0.85 | <.0001 |
| Highest | | | | | | | | |
| education, years | | | | | | | | |
| 1-9 | 30195 | 130632 | 4162 | 31.9 | | Reference | | |
| 10-11 | 33915 | 146503 | 2805 | 19.1 | 0.60 | 0.57 | 0.63 | <.0001 |
| 12+ | 25873 | 111931 | 1684 | 15.0 | 0.46 | 0.43 | 0.48 | <.0001 |
| Birth country | | | | | | | | |
| Sweden | 81974 | 354884 | 7921 | 22.3 | | Reference | | |
| European | 6381 | 27564 | 629 | 22.8 | 1.02 | 0.94 | 1.11 | 0.6033 |
| countries | | | | | | | | |
| Others | 1628 | 6618 | 101 | 15.3 | 0.68 | 0.56 | 0.83 | 0.0001 |
| Income | | | | | | | | |
| Lowest | 22941 | 94587 | 3245 | 34.3 | | Reference | | |
| Middle-Low | 22357 | 96240 | 2660 | 27.6 | 0.81 | 0.77 | 0.85 | <.0001 |
| Middle-High | 22342 | 97804 | 1639 | 16.8 | 0.49 | 0.46 | 0.52 | <.0001 |
| Highest | 22343 | 100435 | 1107 | 11.0 | 0.32 | 0.30 | 0.35 | <.0001 |
| Region | | | | | | | | |
| Big cities | 42634 | 187666 | 3535 | 18.8 | | Reference | | |
| Southern Sweden | 29486 | 128057 | 3030 | 23.7 | 1.26 | 1.20 | 1.32 | <.0001 |
| North Sweden | 17863 | 73343 | 2089 | 28.5 | 1.51 | 1.43 | 1.59 | <.0001 |
| Stage at Diagnosis | | | | | | | | |
| Stage 1 | 43782 | 203721 | 969 | 4.8 | | Reference | | |
| Stage 2 | 25726 | 114529 | 1584 | 13.8 | 2.92 | 2.70 | 3.17 | <.0001 |
| Stage 3 | 11827 | 47288 | 2071 | 43.8 | 9.34 | 8.66 | 10.08 | <.0001 |
| Stage 4 | 8648 | 23528 | 4027 | 171.2 | 37.70 | 35.13 | 40.46 | <.0001 |
| Chronic ischemic | | | | | | | | |
| heart disease | | | | | | | | |
| No | 74741 | 322143 | 6617 | 20.5 | | Reference | | |
| Yes | 15242 | 66924 | 2034 | 30.4 | 1.48 | 1.41 | 1.56 | <.0001 |
| Diabetes | | | | | | | | |
| No | 82169 | 354080 | 7493 | 21.2 | | Reference | | |
| Yes | 7814 | 34987 | 1158 | 33.1 | 1.57 | 1.47 | 1.67 | <.0001 |
| Chronic obstructive | | | | | | | | |
| pulmonary disease | | | | | | | | |
| No | 85045 | 366882 | 7987 | 21.8 | | Reference | | |
| Yes | 4938 | 22185 | 664 | 29.9 | 1.38 | 1.27 | 1.49 | <.0001 |
| Hypertension | | | | | | | | |
| No | 64595 | 269347 | 5469 | 20.3 | | Reference | | |
| Yes | 25388 | 119720 | 3182 | 26.6 | 1.32 | 1.26 | 1.37 | <.0001 |

TABLE 6
Hazard ratios of prostate cancer mortality among patients with post-diagnostic
use of cholera vaccine as compared to patients without cholera vaccination

| | | No. | | | Crude | | | | Adjusted | | | |
|---|---|---|---|---|---|---|---|---|---|---|---|---|
| | No. | Person- | No. | | | | | P- | | | | P- |
| Characteristic | Patients | years | Deaths | IR* | HR** | 95% CI | | value | HR | 95% CI | | value |
| Post-diagnostic use | | | | | | | | | | | | |
| of cholera vaccine | | | | | | | | | | | | |
| No | 89,142 | 383,035 | 8,622 | 22.5 | Reference | | | | Reference | | | |
| Yes | 841 | 6,033 | 29 | 4.8 | 0.33 | 0.23 | 0.47 | <0.0001 | 0.57 | 0.40 | 0.82 | 0.00 |
| Age at Diagnosis | | | | | | | | | | | | |
| <65 | 445 | 3,344 | 9 | 2.7 | 0.45 | 0.23 | 0.86 | 0.02 | 0.57 | 0.30 | 1.11 | 0.09 |
| ≥65 | 396 | 2,689 | 20 | 7.4 | 0.36 | 0.23 | 0.56 | <0.0001 | 0.60 | 0.38 | 0.93 | 0.02 |
| Stage at diagnosis | | | | | | | | | | | | |
| Stages 1 and 2 | 738 | 5,365 | 9 | 1.7 | 0.25 | 0.13 | 0.47 | <0.0001 | 0.54 | 0.28 | 1.04 | 0.06 |
| Stages 3 and 4 | 103 | 668 | 20 | 30.0 | 0.51 | 0.33 | 0.79 | 0.00 | 0.62 | 0.40 | 0.97 | 0.03 |
| Income | | | | | | | | | | | | |
| Lowest | 71 | 499 | 1 | 2.0 | 0.09 | 0.01 | 0.62 | 0.01 | 0.20 | 0.03 | 1.40 | 0.10 |
| Middle-low | 129 | 935 | 2 | 2.1 | 0.12 | 0.03 | 0.46 | 0.01 | 0.20 | 0.05 | 0.82 | 0.02 |
| Middle-high | 213 | 1,544 | 7 | 4.5 | 0.40 | 0.19 | 0.85 | 0.02 | 0.54 | 0.26 | 1.13 | 0.10 |
| Highest | 428 | 3,055 | 19 | 6.2 | 0.80 | 0.51 | 1.26 | 0.32 | 0.73 | 0.46 | 1.16 | 0.18 |

*IR indicates the incidence rate of prostate cancer mortality per 1000 person-years
**HR indicates the hazard ratio, and it was adjusted by age at diagnosis, year of diagnosis, birth country, highest education level, stage at diagnosis, income, region, chronic ischemic heart disease, chronic obstructive pulmonary disease, diabetes, and hypertension.

TABLE 7
Sensitivity analyses of the adjusted HR between post-
diagnostic use of cholera vaccine and the risk of death

| Charac- | Cause-specific mortality | | | | Overall mortality | | | |
|---|---|---|---|---|---|---|---|---|
| teristic | HR | 95% CI | | P-value | HR | 95% CI | | P-value |
| Sensitivity | 0.46 | 0.31 | 0.68 | <0.0001 | 0.54 | 0.43 | 0.69 | <0.0001 |
| analysis 1 | | | | | | | | |
| Sensitivity | 0.50 | 0.33 | 0.76 | 0.001 | N/A | | | |
| analysis 2 | | | | | | | | |
| Sensitivity | 0.49 | 0.31 | 0.74 | 0.001 | 0.53 | 0.39 | 0.71 | <0.0001 |
| analysis 3 | | | | | | | | |
| Sensitivity | 1.52 | 1.35 | 1.71 | <0.0001 | 1.46 | 1.36 | 1.57 | <0.0001 |
| analysis 4 | | | | | | | | |
| Sensitivity | 0.54 | 0.35 | 0.83 | <0.0001 | 0.53 | 0.39 | 0.70 | <0.0001 |
| analysis 5 | | | | | | | | |

Sensitivity analysis 1: one year of latency time windows between cholera vaccination and mortality.
Sensitivity analysis 2: Fine & Gray competing risk model.
Sensitivity analysis 3: Removing patients with prostate cancer who were identified by screening.
Screening analysis 4: Patients with prostate cancer who received antimalarial treatment as compared to non-exposed cases.
Screening analysis 5: Using patients without vaccination and matched by propensity score as the reference.

### Risk of Death Due to all the Causes

Table 8 shows the risk of overall mortality among patients with prostate cancer who used cholera vaccine. The overall mortality rate was 9.4 per 1000 person-years for patients using cholera vaccine, whereas the rate was 48.3 for those who do not use cholera vaccine. A decreased overall mortality rate was found among patients using cholera vaccine, as compared with patients without cholera vaccination use, with a crude HR of 0.27 (95% CI 0.21-0.35) and an adjusted HR of 0.53 (95% CI 0.41-0.69). The decreased mortality rate was largely consistent, irrespective of age at diagnosis, clinical stage at diagnosis, and disposable income.

TABLE 8
Hazard ratios of overall mortality among patients with post-diagnostic
use of cholera vaccine as compared to patients without cholera vaccination

| | | No. | | | Crude | | | | Adjusted | | | |
|---|---|---|---|---|---|---|---|---|---|---|---|---|
| | No. | Person- | No. | | | | | P- | | | | P- |
| Characteristic | Patients | years | Deaths | IR* | HR** | 95% CI | | value | HR | 95% CI | | value |
| Post-diagnostic use | | | | | | | | | | | | |
| of cholera vaccine | | | | | | | | | | | | |
| No | 89,142 | 383,035 | 18,496 | 48.3 | Reference | | | | Reference | | | |
| Yes | 841 | 6,033 | 57 | 9.4 | 0.27 | 0.21 | 0.35 | <0.0001 | 0.53 | 0.41 | 0.69 | <0.0001 |
| Age at Diagnosis | | | | | | | | | | | | |
| <65 | 445 | 3,344 | 18 | 5.4 | 0.46 | 0.29 | 0.74 | 0.001 | 0.61 | 0.38 | 0.97 | 0.04 |
| ≥65 | 396 | 2,689 | 39 | 14.5 | 0.29 | 0.21 | 0.40 | <0.0001 | 0.53 | 0.39 | 0.73 | 0.001 |
| Stage at diagnosis | | | | | | | | | | | | |
| Stages 1 and 2 | 738 | 5,365 | 34 | 6.3 | 0.27 | 0.19 | 0.37 | <0.0001 | 0.58 | 0.41 | 0.81 | 0.001 |
| Stages 3 and 4 | 103 | 668 | 23 | 34.5 | 0.36 | 0.24 | 0.53 | <0.0001 | 0.52 | 0.35 | 0.79 | 0.002 |
| Income | | | | | | | | | | | | |
| Lowest | 71 | 499 | 4 | 8.0 | 0.14 | 0.05 | 0.38 | <0.0001 | 0.37 | 0.14 | 0.99 | 0.04 |
| Middle-low | 129 | 935 | 9 | 9.6 | 0.21 | 0.11 | 0.41 | <0.0001 | 0.38 | 0.20 | 0.73 | 0.003 |
| Middle-high | 213 | 1,544 | 16 | 10.4 | 0.42 | 0.25 | 0.68 | 0.001 | 0.59 | 0.36 | 0.97 | 0.03 |
| Highest | 428 | 3,055 | 28 | 9.2 | 0.53 | 0.36 | 0.77 | 0.001 | 0.58 | 0.40 | 0.84 | 0.003 |

### Sensitivity Analyses

Sensitivity analyses were performed by redefining exposure as one year after the administration of cholera vaccine and these gave similar results (Table 9). Accounting for competing risks and removing cases identified by screening did not change the observed association. Patients who received antimalarial medications showed an increased cause-specific and overall mortality rate, as compared to patients who did not use antimalarial medications. Only 90 of them received both cholera vaccination and antimalarial medications (Table 10).

TABLE 9
Associations of prostate cancer mortality with clinical and demographic characteristics

| | | No. | | | | | | |
|---|---|---|---|---|---|---|---|---|
| | No. | person- | No. | | | | | |
| Characteristic | patients | years | deaths | IR | HR | 95% CI | | P-value |
| Age at diagnosis | | | | | | | | |
| <60 | 12,253 | 59,981 | 514 | 8.6 | Reference | | | |
| 60-64 | 16,192 | 80,092 | 762 | 9.5 | 1.11 | 0.99 | 1.24 | 0.067 |
| 65-69 | 21,255 | 93,194 | 1,183 | 12.7 | 1.48 | 1.33 | 1.64 | <0.001 |
| 70+ | 40,283 | 155,799 | 6192 | 39.7 | 4.62 | 4.22 | 5.05 | <0.001 |
| Year at diagnosis | | | | | | | | |
| 2005-2009 | 37,512 | 248,699 | 5741 | 23.1 | Reference | | | |
| 2010-2014 | 52,471 | 140,368 | 2,910 | 20.7 | 0.81 | 0.77 | 0.85 | <0.0001 |
| Highest | | | | | | | | |
| education-years | | | | | | | | |
| 1-9 | 30,195 | 130,632 | 4,162 | 31.9 | Reference | | | |
| 10-11 | 33,915 | 146,503 | 2,805 | 19.1 | 0.60 | 0.57 | 0.63 | <0.001 |
| 12+ | 25,873 | 111,931 | 1,684 | 15.0 | 0.46 | 0.43 | 0.48 | <0.001 |
| Birth country | | | | | | | | |
| Sweden | 81,974 | 354,884 | 7,921 | 22.3 | Reference | | | |
| European | 6,381 | 27,564 | 629 | 22.8 | 1.02 | 0.94 | 1.11 | 0.6033 |
| countries | | | | | | | | |
| Others | 1628 | 6618 | 101 | 15.3 | 0.68 | 0.56 | 0.83 | 0.0001 |
| Income | | | | | | | | |
| Lowest | 22,941 | 94,587 | 3,245 | 34.3 | Reference | | | |
| Middle-low | 22,357 | 96,240 | 2,660 | 27.6 | 0.81 | 0.77 | 0.85 | <0.001 |
| Middle-high | 22,342 | 97,804 | 1,639 | 16.8 | 0.49 | 0.46 | 0.52 | <0.001 |
| Highest | 22,343 | 100,435 | 1,107 | 11.0 | 0.32 | 0.30 | 0.35 | <0.001 |
| Region | | | | | | | | |
| Big cities | 42,634 | 187,666 | 3,532 | 18.8 | Reference | | | |
| Southern Sweden | 29,486 | 128,057 | 3,030 | 23.7 | 1.26 | 1.20 | 1.32 | <0.001 |
| Northern Sweden | 17,863 | 73,343 | 2,089 | 28.5 | 1.51 | 1.43 | 1.59 | <0.001 |
| Stage at diagnosis | | | | | | | | |
| Stage 1 | 43,782 | 203,721 | 969 | 4.8 | Reference | | | |
| Stage 2 | 25,726 | 114,529 | 1,584 | 13.8 | 2.92 | 2.70 | 3.17 | <0.001 |
| Stage 3 | 11,827 | 47,288 | 2,071 | 43.8 | 9.34 | 8.66 | 10.08 | <0.001 |
| Stage 4 | 8,648 | 23,528 | 4,027 | 171.2 | 37.70 | 35.13 | 40.46 | <0.001 |
| Chronic ischemic | | | | | | | | |
| heart disease | | | | | | | | |
| No | 74,741 | 322,143 | 6,617 | 20.5 | Reference | | | |
| Yes | 15,242 | 66,924 | 2,034 | 30.4 | 1.48 | 1.41 | 1.56 | <0.001 |
| Diabetes | | | | | | | | |
| No | 82,169 | 354,080 | 7,493 | 21.2 | Reference | | | |
| Yes | 7,814 | 34,987 | 1,158 | 33.1 | 1.57 | 1.47 | 1.67 | <0.001 |
| Chronic obstructive | | | | | | | | |
| pulmonary disease | | | | | | | | |
| No | 85,045 | 366,882 | 7,987 | 21.8 | Reference | | | |
| Yes | 25,388 | 119,720 | 3,182 | 26.6 | 1.32 | 1.26 | 1.37 | <0.001 |
| Hypertension | | | | | | | | |
| No | 64,595 | 269,347 | 5,469 | 20.3 | Reference | | | |
| Yes | 25,388 | 119,720 | 3,182 | 26.6 | 1.32 | 1.26 | 1.37 | <0.001 |

TABLE 10
Cross tabulation of exposure to cholera
vaccine and antimalarial medications

| | | Antimalarial medication | | | |
|---|---|---|---|---|---|
| | | Yes | | No | |
| | | Number | % | Number | % |
| Cholera vaccine | Yes | 90 | 0.10 | 751 | 0.83 |
| | No | 3549 | 3.94 | 85,593 | 95.12 |

### Discussion

This retrospective cohort study is the first nationwide population-based study to explore whether use of cholera vaccine is associated with a reduced mortality rate in patients with prostate cancer. Patients with prostate cancer who had used cholera vaccine after their cancer diagnosis were found to have had a 43% decrease in prostate cancer mortality and a 47% decrease in overall mortality, as compared to patients who did not use cholera vaccine. The decreased mortality rate associated with cholera vaccination was largely consistent, irrespective of the patient's age or tumor stage at diagnosis, and other potential cofounders, including disposable income. Mortality reductions were also noted in several sensitivity analyses. Our findings suggest that cholera vaccine is associated with a reduced mortality rate in patients with prostate cancer³⁰⁻³⁷.

Studies examining the potential benefits of non-anticancer drugs or agents to patients with cancer, also called drug repurposing, are an attractive strategy for both academics and clinicians.³⁸ Such drugs, if successful, may offer additional treatment options to patients with cancer. A number of observational studies have been carried out to investigate the association between the use of non-anticancer drugs, such as statin and metformin, and different prostate cancer outcomes.³⁹⁻⁴⁵ However, several potential biases could affect the observed associations and lead to inconsistent results. Common biases in these observation studies include immortal time bias, indication bias, health user bias, as well as and consideration of latency time windows between the use of non-anticancer drugs and outcomes.⁴⁶ In this population-based study, we have examined whether the use of cholera vaccine might be associated with a reduced mortality rate in patients with prostate cancer by taking into account all of the biases listed above. By using time-dependent Cox regression analyses, we could control the confounding by immortal time bias (main analysis). In addition, the influence of latency time on the observed associations was explored by defining the exposure to cholera vaccine as 1 year after vaccination (Sensitivity analysis 1). To control health user bias, we removed all prostate cancer cases that had been detected by screening (Sensitivity analysis 3), as men who undertook PSA screening might be associated with a high education and many types of healthy behavior.⁴⁷ In addition, we used patients without vaccination and matched them by a propensity score (no difference for all the clinical and demographic factors listed in Table 4, as compared to the study cohorts) as the reference; the data were largely similar. Furthermore, confounding by indication could not be totally ruled out, as we have no indications as to why some patients with prostate cancer received a vaccination for cholera. To control for this indication bias, we examined the mortality rate in patients with prostate cancer who used antimalarial medications; our hypothesis being that most Swedes who received either antimalarial medications or cholera vaccine might have traveled abroad (Sensitivity analysis 4). Although this study cannot eliminate all the confounding by unmeasured or misclassified prognostic factors that are associated with prostate cancer mortality, this study did give concrete evidence that use of cholera vaccine was associated with a risk reduction of cause-specific and overall mortality, as both the main analyses and several sensitivity analyses gave consistent results.

The underlying mechanisms are largely unknown and further studies are required to explore the origins of these mechanisms. Previous animal studies have reported that cholera vaccine might have an antitumor function.³⁰⁻³⁷ CTB can bind to the ganglioside on mammalian cells and is used as a component of oral cholera vaccine in Sweden.⁵ CTB can induced an anti-inflammatory effect and regulate T-cell responses⁶⁰. Animal studies showed that the administration of recombinant CTB can increase some immune cell populations, such as NK cells and macrophages.^{26,48} In addition, some animal studies found that Th1 cells and CD8+ T cells can be promoted to prevent tumor growth after CTB vaccination inflates them.⁴⁹ In addition, available evidence suggests that the intestinal microbiota can affect how an individual response to cholera vaccine.⁵⁰ Nicaraguan children who received an oral cholera vaccine have shown reduced antibody responses as compared to Swedish children.⁵⁰ A similar finding was noted among individuals who received a vaccination of Shigella flexneri with different immune responses among Bangladeshi adults and children, as compared to North American individuals.⁵¹ These data suggest that the composition of intestinal microbiota may be a determining factor of vaccine efficacy, although other factors, such as socioeconomic conditions, nutritional status, and host genetics might also play a role. It remains unknown whether intestinal microbiota might be altered after oral cholera vaccination. Excessive bacterial growth was observed in the small intestine among children who received cholera vaccine,⁵² which suggests that the intestinal microbiota might be altered after oral cholera vaccination. As intestinal microbiota might play an important role regarding antitumor immunity,^{53,54} cholera vaccination might protect against the progression of prostate cancer by alternating intestinal microbiota. Such knowledge is still lacking but highly needed in further studies. A study did find that cholera vaccination was associated with a lower mortality rate in patients with prostate cancer. Further studies, including well-designed cohort studies and randomized clinical trials, are warranted to confirm our research findings and to draw a causal relationship. In addition, whether the observed low mortality in patients who received cholera vaccination was due to improve immune function or altered intestinal microbiota should be explored further.

An important strength of this population-based study is that it has taken advantage of several nationwide registers in Sweden, which cover the whole Swedish population at a national level. The prospective study design and the completeness of the follow-up of patients are other major advantages of the present study. By using nationwide registers, our study could eliminate the recall bias and could minimize selection bias and misclassification. Some demographic factors and several clinical factors, such as the age of the patient at the time of diagnosis and tumor stage, which are the most significant factors that affect cancer mortality, could be identified from these registers and are included in the regression models. One limitation of this study is that the information on some individual-level risk factors, such as the PSA value, Gleason score, medical treatments, smoking status, and dietary factors are not available in our database, which may have partly confounded our conclusion. However, we could adjust the clinical stage at diagnosis in our regression models, which was the strongest prognostic factor for prostate cancer. In addition, Sweden is well-known for its universal healthcare for all Swedish citizens that is provided at a minimal cost. Discrepancy in medical treatment of prostate cancer is uncommon, as treatment decisions are made mainly based on clinical conditions instead of socioeconomic status. However, a recent study found that socioeconomic disparities in the management of men with a high risk of prostate cancer also exist in Sweden, but the discrepancy was minimal.⁵⁵ To account for the confounding by socioeconomic disparities in the management of prostate cancer, we have adjusted for place at living, disposable income, and education level in our regression model, which may partly minimize the confounding by these unmeasured factors.^{56,57} In addition, we also stratified the analyses by disposable income, and the results were largely consistent. By using patients without vaccination and matching them by a propensity score as the reference, the observed findings were still significant, thus suggesting that the observed findings due to a discrepancy in socioeconomic status might be minimal.

In summary, this population-based study shows that the use of cholera vaccine is associated with a decreased mortality in patients with prostate cancer. Risk reduction was largely consistent, irrespective of the age of the patients at the time of diagnosis and clinical stage, The findings from this study need to be confirmed by further randomized controlled studies to exclude the possibility of chance findings.

### REFERENCES

24. De Angelis, R. et al. Cancer survival in Europe 1999-2007 by country and age: results of EUROCARE-5-a population-based study. Lancet Oncol. 15,23-34 (2014).
25. Jemal, A. et al. Annual report to the nation on the status of cancer, 1975-2014, featuring survival. J. Natl Cancer. Inst. 109, djx030 (2017).
26. Doulberis, M. et al. Cholera-toxin suppresses carcinogenesis in a mouse model of inflammation-driven sporadic colon cancer. Carcinogenesis 36, 280-290 (2015).
27. Bharati, K. & Ganguly, N. K. Cholera toxin: a paradigm of a multifunctional protein. Indian J. Med. Res. 133, 179-187 (2011).
28. Baldauf, K. J. et al. Cholera toxin B: one subunit with many pharmaceutical applications. Toxins 7, 974-996 (2015).
29. Chen, P. et al. Restraint of proinflammatory cytokine biosynthesis by mitogen-activated protein kinase phosphatase-1 in lipopolysaccharide-stimulated macrophages. J. Immunol. 169, 6408-6416 (2002).
30. Ji, J., Sundquist, J., & Sundquist, K. Cholera vaccine use is associated with a reduced risk of death in patients with colorectal cancer: a population-based study. Gastroenterology 154,86-92.el (2018).
31. Isomura, I. et al. Recombinant cholera toxin B subunit activates dendritic cells and enhances antitumor immunity. Microbiol. Immunol. 49,79-87 (2005).
32. Lavelle, E. C. et al. Effects of cholera toxin on innate and adaptive immunity and its application as an immunomodulatory agent. J. Leukoc. Biol. 75, 756-763 (2004).
33. Wiedinger, K., Romlein, H. & Bitsaktsis, C. Cholera toxin B induced activation of murine macrophages exposed to a fixed bacterial immunogen. Ther. Adv. Vaccines 3, 155-163 (2015).
34. Block, M. I., Alexander, H. R., & Norton, J. A. Cholera toxin pretreatment protects against tumor necrosis factor lethality without compromising tumor response to therapy. Arch. Surg. 127 1330-1334 (1992).
35. Wakabayashi, A. et al. Development of antitumor immunity by oral vaccination with tumor antigen and cholera toxin. J. Nippon Med. Sch. 77, 50-52 (2010).
36. Kaur, G. et al. Growth inhibition by cholera toxin of human lung carcinoma cell lines: correlation with GM1 ganglioside expression. Cancer Res. 52, 3340-3346 (1992).
37. Xia, Q. et al. Cholera toxin inhibits human hepatocarcinoma cell proliferation in vitro via suppressing ATX/LPA axis. Acta Pharmacol. Sin. 32, 1055-1062 (2011).
38. Bertolini, F., Sukhatme, V. P. & Bouche, G. Drug repurposing in oncology-patient and health systems opportunities. Nat. Rev. Clin. Oncol. 12, 732-742 (2015).
39. Murtola, T. J. et al. Statin use and prostate cancer survival in the finish randomized study of screening for prostate cancer. Eur. Urol. Focus 3, 212-220 (2017).
40. Keskivali, T. et al. Statin use and risk of disease recurrence and death after radical prostatectomy. Prostate 76, 469-478 (2016).
41. Zhong, S. et al. Statin use and mortality in cancer patients: systematic review and meta-analysis of observational studies. Cancer Treat. Rev. 41,554-567 (2015).
42. Sun, L. M. et al. Statin use reduces prostate cancer all-cause mortality: a nationwide population-based cohort study. Medicine 94, e1644 (2015).
43. Bensimon, L. et al. The use of metformin in patients with prostate cancer and the risk of death. Cancer Epidemiol. Biomarkers Prev. 23, 2111-2118 (2014).
44. Margel, D. et al. Metformin use and all-cause and prostate cancer-specific mortality among men with diabetes. J. Clin. Oncol. 31, 3069-3075 (2013).
45. Franciosi, M. et al. Metformin therapy and risk of cancer in patients with type 2 diabetes: systematic review. PLoS ONE 8, e71583 (2013).
46. Yu, O. et al. Use of statins and the risk of death in patients with prostate cancer. J. Clin. Oncol. 32,5-11 (2014).
47. Karlsen, R. V. et al. PSA testing without clinical indication for prostate cancer in relation to socio-demographic and clinical characteristics in the Danish Diet, Cancer and Health Study. Acta Oncol. 52, 1609-1614 (2013).
48. Baldauf, K. J. et al. Oral administration of a recombinant cholera toxin B subunit promotes mucosal healing in the colon. Mucosal Immunol. 10, 887-900 (2017).
49. Lu, W. et al. Cytotoxic T cell responses are enhanced by antigen design involving the presentation of MUC1 peptide on cholera toxin B subunit. Oncotarget 6, 34537-34548 (2015).
50. Hallander, H. O. et al. Calibrated serological techniques demonstrate significant different serum response rates to an oral killed cholera vaccine between Swedish and Nicaraguan children. Vaccine 21, 138-145 (2002).
51. World Health Organization. Future needs and directions for Shigella vaccines (Besoins et orientations futurs en matière de vaccins anti-Shigella). Wkly. Epidemiol. Rec. (Relev. Epidemiol. Hebd.) 81,51-58 (2006).
52. Lagos, R. et al. Effect of small bowel bacterial overgrowth on the immunogenicity of single-dose live oral cholera vaccine CVD 103-HgR. J. Infect. Dis. 180, 1709-1712 (1999).
53. Amirian, E. S. et al. Potential role of gastrointestinal microbiota composition in prostate cancer risk. Infect. Agents Cancer 8, 42 (2013).
54. Golombos, D. M. et al. The role of gut microbiome in the pathogenesis of prostate cancer: a prospective, pilot study. Urology 111, 122-128 (2018).
55. Berglund, A. et al. Differences according to socioeconomic status in the management and mortality in men with high risk prostate cancer. Eur. J. Cancer 48,75-84 (2012).
56. Eek, F. et al. Differences in socioeconomic and gender inequalities in tobacco smoking in Denmark and Sweden; a cross sectional comparison of the equity effect of different public health policies. BMC Public Health 10, 9 (2010).
57. Ax, E. et al. Dietary patterns in Swedish adults; results from a national dietary survey. Br. J. Nutr. 115,95-104 (2016).
58. Ji, J., Sundquist, K. & Sundquist, J. A population-based study of hepatitis D virus as potential risk factor for hepatocellular carcinoma. J. Natl. Cancer Inst. 104, 790-792 (2012).
59. Hemminki, K. et al. Risk of familial breast cancer is not increased after pregnancy. Breast Cancer Res. Treat. 108, 417-420, (2008).
60. Sobin, L. H., Gospodarowicz, M. K. & Wittekind, C. (eds). TNM Classification of Malignant Tumours, 7th edn (Wiley-Blackwell, 2011).
61. Chubak, J. et al. Threats to validity of nonrandomized studies of postdiagnosis exposures on cancer recurrence and survival. J. Natl. Cancer Inst. 105, 1456-1462 (2013).
62. Latouche, A., Porcher, R. & Chevret, S. A note on including time-dependent covariate in regression model for competing risks data. Biom. J. 47, 807-814 (2005).

### Preferred Aspects

### What is Aspected is:

A1. A method of treating or preventing cancer, comprising

administering to a subject in need thereof a therapeutically effective amount of

(1) a composition comprising lipopolysaccharide (LPS) from at least one strain of Vibrio cholerae,

(2) a composition comprising cholera toxin, or

(3) a composition comprising a combination of lipopolysaccharide (LPS) from at least one strain of Vibrio cholerae and cholera toxin.

A2. The method of aspect A1, wherein the cholera toxin is a subunit of cholera toxin.
A3. The method of aspect A2, wherein the subunit of cholera toxin is cholera toxin subunit B.
A4. The method of any one of aspects A2-A3, wherein the cholera toxin is recombinantly produced.
A5. The method of any one of aspects A1-A4, wherein the cholera toxin is from strains belonging to V. cholerae Inaba classical biotype, V. cholerae Inaba El Tor biotype, and V. cholerae Ogawa classical biotype.
A6. The method of any one of aspects A1-A5, wherein the at least one strain of V. cholerae is of serotype O1.
A7. The method of aspect A6, wherein the at least one strain of V. cholerae is selected from the group consisting of V. cholerae Inaba classical biotype, V. cholerae Inaba El Tor biotype, and Ogawa classical biotype.
A8. The method of aspect A6 or A7, wherein the composition comprises LPS from three strains of V. cholerae.
A9. The method of aspect A8, wherein the LPS is from V. cholerae Inaba classical biotype, V. cholerae Inaba El Tor biotype, and V. cholerae Ogawa classical biotype.
A10. The method of anyone of aspects A1-A9, wherein the LPS is in the form of whole V. cholerae bacteria.
A11. The method of aspect A10, wherein the whole V. cholerae bacteria are killed/inactivated and/or heat-inactivated.
A12. The method of aspect A10, wherein the whole V. cholerae bacteria are attenuated by deletion of at least a portion of a nucleic acid sequence encoding the A subunit of cholera toxin.
A13. The method of anyone of aspects A10-A12, wherein the LPS is in the form of whole V. cholerae bacteria of three strains of V. cholerae.
A14. The method of aspect A13, wherein the bacteria of at least one of the strains of V. cholerae are killed/inactivated.
A15. The method of aspect A14, wherein the bacteria of at least one of the strains of V. cholerae are heat-inactivated.
A16. The method of aspect A14 or A15, wherein the bacteria of at least one of the strains of V. cholerae are formalin-inactivated.
A17. The method of anyone of aspects A1-A16, further comprising administering an acid-neutralizing agent.
A18. The method of aspect A17, wherein the acid-neutralizing agent is a bicarbonate buffer.
A19. The method of anyone of aspects A1-A18, wherein the composition is administered to the subject at least twice.
A20. The method of anyone of aspects A1-A19, wherein the administration is oral administration or parenteral administration.
A21. The method of anyone of aspects A1-A20, wherein the subject has or is predisposed to have a cancer characterized by chronic inflammation or by expression of GM1 ganglioside receptors.
A22. The method of anyone of aspects A1-A21, wherein the subject has or is predisposed to have colorectal cancer, prostate cancer, bladder cancer, small cell lung cancer, renal cancer, cervical cancer, or lymphoma.
A23. The method of anyone of aspects A1-A22, wherein the subject underwent surgery to remove cancerous tissue and/or received chemotherapy.
A24. The method of anyone of aspects A1-A23, further comprising administering one or more additional therapeutic agent.
A25. The method of anyone of aspects A1-A24, further comprising administering an immune checkpoint inhibitor, such as e.g. a monoclonal antibody against PD-1, PD-L1, and/or CTLA-4.
A26. The method of any one of aspects A1-A25, further comprising administering an EGFR drug and/or a VEGF drug, such as e.g. bevacizumab.
B1. A method of enhancing survival of a subject having cancer, comprising

administering to a subject in need thereof a therapeutically effective amount of

(1) a composition comprising lipopolysaccharide (LPS) from at least one strain of Vibrio cholerae,

(2) a composition comprising cholera toxin, or

(3) a composition comprising lipopolysaccharide (LPS) from at least one strain of Vibrio cholerae and cholera toxin.

B2. The method of aspect B1, wherein the cholera toxin is a subunit of cholera toxin.
B3. The method of aspect B2, wherein the subunit of cholera toxin is cholera toxin subunit B.
B4. The method of any one of aspects B1-B3, wherein the cholera toxin is recombinantly produced.
B4.1 The method of any one of aspects B1-B4, wherein the cholera toxin is from strains belonging to V. cholerae Inaba classical biotype, V. cholerae Inaba El Tor biotype, and V. cholerae Ogawa classical biotype.
B5. The method of any one of aspects B1-B4.1, wherein the at least one strain of V. cholerae is of serotype O1.
B6. The method of aspect B5, wherein the at least one strain of V. cholerae is selected from the group consisting of V. cholerae Inaba classical biotype, V. cholerae Inaba El Tor biotype, and Ogawa classical biotype.
B7. The method of aspect B5 or B6, wherein the composition comprises LPS from three strains of V. cholerae.
B8. The method of aspect B7, wherein the LPS is from V. cholerae Inaba classical biotype, V. cholerae Inaba El Tor biotype, and Ogawa classical biotype.
B9. The method of any one of aspects B1-B8, wherein the LPS is in the form of whole V. cholerae bacteria.
B10. The method of aspect B9, wherein the whole V. cholerae bacteria are killed/inactivated and/or heat-inactivated.
B11. The method of aspect B9, wherein the whole V. cholerae bacteria are attenuated by deletion of at least a portion of a nucleic acid sequence encoding the A subunit of cholera toxin.
B12. The method of anyone of aspects B9-B11, wherein the LPS is in the form of whole V. cholerae bacteria of three strains of V. cholerae.
B13. The method of aspect B12, wherein the bacteria of at least one of the strains of V. cholerae are killed/inactivated.
B14. The method of aspect B13, wherein the bacteria of at least one of the strains of V. cholerae are heat-inactivated.
B15. The method of aspect B13 or B14, wherein the bacteria of at least one of the strains of V. cholerae are formalin-inactivated.
B16. The method of anyone of aspects B1-B15, further comprising administering an acid-neutralizing agent.
B17. The method of aspect B16, wherein the acid-neutralizing agent is a bicarbonate buffer.
B18. The method of anyone of aspects B1-B17, wherein the composition is administered to the subject at least twice.
B19. The method of anyone of aspects B1-B18, wherein the administration is oral administration or parenteral administration.
B20. The method of anyone of aspects B1-B19, wherein the subject has or is predisposed to have a cancer characterized by chronic inflammation or by expression of GM1 ganglioside receptors.
B21. The method of anyone of aspects B1-B20, wherein the subject has or is predisposed to have colorectal cancer, prostate cancer, bladder cancer, small cell lung cancer, renal cancer, cervical cancer, or lymphoma.
B22. The method of anyone of aspects B1-B21, wherein the subject has undergone a surgical procedure to remove cancerous tissue and/or has received chemotherapy.
B23. The method of anyone of aspects B1-B22, further comprising administering one or more additional therapeutic agent.
B24. The method of anyone of aspects B1-B23, further comprising administering an immune checkpoint inhibitor, such as e.g. a monoclonal antibody against PD-1, PD-L1 and/or CTLA-4.
B25. The method of anyone of aspects B1-B24, further comprising administering an EGFR drug and/or a VEGF drug, such as e.g. bevacizumab.
C1. A pharmaceutical composition comprising

(1) lipopolysaccharide (LPS) from at least one strain of Vibrio cholerae,

(2) cholera toxin, or

(3) a combination of lipopolysaccharide (LPS) from at least one strain of Vibrio cholerae and cholera toxin, for use in the treatment of cancer.

D1. Use of a composition comprising

(1) lipopolysaccharide (LPS) from at least one strain of Vibrio cholerae,

(2) cholera toxin, or

(3) a combination of lipopolysaccharide (LPS) from at least one strain of Vibrio cholerae and cholera toxin, for the treatment of cancer.

What is claimed is:

1. A method of treating or preventing cancer, comprising
administering to a subject in need thereof a therapeutically effective amount of
(1) a composition comprising lipopolysaccharide (LPS) from at least one strain of Vibrio cholerae,
(2) a composition comprising cholera toxin, or
(3) a composition comprising a combination of lipopolysaccharide (LPS) from at least one strain of Vibrio cholerae and cholera toxin.

2. The method of claim 1, wherein the cholera toxin is a subunit of cholera toxin.

3. The method of claim 2, wherein the subunit of cholera toxin is cholera toxin subunit B.

4. The method of any one of claims 2-3, wherein the cholera toxin is recombinantly produced.

5. The method of any one of claims 1-4, wherein the cholera toxin is from strains belonging to V. cholerae Inaba classical biotype, V. cholerae Inaba El Tor biotype, and V. cholerae Ogawa classical biotype.

6. The method of any one of claims 1-5, wherein the at least one strain of V. cholerae is of serotype O1.

7. The method of claim 6, wherein the at least one strain of V. cholerae is selected from the group consisting of V. cholerae Inaba classical biotype, V. cholerae Inaba El Tor biotype, and Ogawa classical biotype.

8. The method of claim 6 or 7, wherein the composition comprises LPS from three strains of V. cholerae.

9. The method of claim 8, wherein the LPS is from V. cholerae Inaba classical biotype, V. cholerae Inaba El Tor biotype, and V. cholerae Ogawa classical biotype.

10. The method of any one of claims 1-9, wherein the LPS is in the form of whole V. cholerae bacteria.

11. The method of claim 10, wherein the whole V. cholerae bacteria are killed/inactivated and/or heat-inactivated.

12. The method of claim 10, wherein the whole V. cholerae bacteria are attenuated by deletion of at least a portion of a nucleic acid sequence encoding the A subunit of cholera toxin.

13. The method of any one of claims 10-12, wherein the LPS is in the form of whole V. cholerae bacteria of three strains of V. cholerae.

14. The method of claim 13, wherein the bacteria of at least one of the strains of V. cholerae are killed/inactivated.

15. The method of claim 14, wherein the bacteria of at least one of the strains of V. cholerae are heat-inactivated.

16. The method of claim 14 or 15, wherein the bacteria of at least one of the strains of V. cholerae are formalin-inactivated.

17. The method of any one of claims 1-16, further comprising administering an acid-neutralizing agent.

18. The method of claim 17, wherein the acid-neutralizing agent is a bicarbonate buffer.

19. The method of any one of claims 1-18, wherein the composition is administered to the subject at least twice.

20. The method of any one of claims 1-19, wherein the administration is oral administration or parenteral administration.

21. The method of any one of claims 1-20, wherein the subject has or is predisposed to have a cancer characterized by chronic inflammation or by expression of GM1 ganglioside receptors.

22. The method of any one of claims 1-21, wherein the subject has or is predisposed to have colorectal cancer, prostate cancer, bladder cancer, small cell lung cancer, renal cancer, cervical cancer, or lymphoma.

23. The method of any one of claims 1-22, wherein the subject underwent surgery to remove cancerous tissue and/or received chemotherapy.

24. The method of any one of claims 1-23, further comprising administering one or more additional therapeutic agent.

25. The method of any one of claims 1-24, further comprising administering an immune checkpoint inhibitor, such as e.g. a monoclonal antibody against PD-1, PD-L1, and/or CTLA-4.

26. The method of any one of claims 1-25, further comprising administering an EGFR drug and/or a VEGF drug, such as e.g. bevacizumab.

27. A method of enhancing survival of a subject having cancer, comprising
administering to a subject in need thereof a therapeutically effective amount of
(1) a composition comprising lipopolysaccharide (LPS) from at least one strain of Vibrio cholerae,
(2) a composition comprising cholera toxin, or
(3) a composition comprising lipopolysaccharide (LPS) from at least one strain of Vibrio cholerae and cholera toxin.

28. The method of claim 27, wherein the cholera toxin is a subunit of cholera toxin.

29. The method of claim 28, wherein the subunit of cholera toxin is cholera toxin subunit B.

30. The method of any one of claims 27-29, wherein the cholera toxin is recombinantly produced.

31. The method of any one of claims 27-30, wherein the cholera toxin is from strains belonging to V. cholerae Inaba classical biotype, V. cholerae Inaba El Tor biotype, and V. cholerae Ogawa classical biotype.

32. The method of any one of claims 27-31, wherein the at least one strain of V. cholerae is of serotype O1.

33. The method of claim 32, wherein the at least one strain of V. cholerae is selected from the group consisting of V. cholerae Inaba classical biotype, V. cholerae Inaba El Tor biotype, and Ogawa classical biotype.

34. The method of claim 32 or 33, wherein the composition comprises LPS from three strains of V. cholerae.

35. The method of claim 34, wherein the LPS is from V. cholerae Inaba classical biotype, V. cholerae Inaba El Tor biotype, and Ogawa classical biotype.

36. The method of any one of claims 27-35, wherein the LPS is in the form of whole V. cholerae bacteria.

37. The method of claim 36, wherein the whole V. cholerae bacteria are killed/inactivated and/or heat-inactivated.

38. The method of claim 37, wherein the whole V. cholerae bacteria are attenuated by deletion of at least a portion of a nucleic acid sequence encoding the A subunit of cholera toxin.

39. The method of any one of claims 36-38, wherein the LPS is in the form of whole V. cholerae bacteria of three strains of V. cholerae.

40. The method of claim 39, wherein the bacteria of at least one of the strains of V. cholerae are killed/inactivated.

41. The method of claim 40, wherein the bacteria of at least one of the strains of V. cholerae are heat-inactivated.

42. The method of claim 40 or 41, wherein the bacteria of at least one of the strains of V. cholerae are formalin-inactivated.

43. The method of any one of claims 27-42, further comprising administering an acid-neutralizing agent.

44. The method of claim 43, wherein the acid-neutralizing agent is a bicarbonate buffer.

45. The method of any one of claims 26-44, wherein the composition is administered to the subject at least twice.

46. The method of any one of claims 27-45, wherein the administration is oral administration or parenteral administration.

47. The method of any one of claims 27-46, wherein the subject has or is predisposed to have a cancer characterized by chronic inflammation or by expression of GM1 ganglioside receptors.

48. The method of any one of claims 27-47, wherein the subject has or is predisposed to have colorectal cancer, prostate cancer, bladder cancer, small cell lung cancer, renal cancer, cervical cancer, or lymphoma.

49. The method of any one of claims 27-48, wherein the subject has undergone a surgical procedure to remove cancerous tissue and/or has received chemotherapy.

50. The method of any one of claims 27-49, further comprising administering one or more additional therapeutic agent.

51. The method of any one of claims 27-50, further comprising administering an immune checkpoint inhibitor, such as e.g. a monoclonal antibody against PD-1, PD-L1 and/or CTLA-4.

52. The method of any one of claims 27-51, further comprising administering an EGFR drug and/or a VEGF drug, such as e.g. bevacizumab.
